SEQUENCE LISTING (3 sequences)

SEQ ID NO: 1 - protein, 391 aa, Homo sapiens
MPPSGLRLLPLLLPLLWLLVLTPGPPAAGLSTCKTIDMELVKRKRIEAIRGQILSKLRIASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRRLKLKVEQHVELYQKYSNNSWRYLSNRLLAPSDSPEWLSFDVTGVVRQWLSRGGEIEGFRISAHCSCDSRDNTLQVDINGFTTGRRGDLTAIHGMNRPFLLLMATPLERAQHLQSSRHRRALDTNYCFSSTEKNCCVRQLYIDFRKDLGWKWIHEPKGYHANFCLGPCPYIWSIDTQYSKVLALYNQHNPGASAAPCCVPQALEPLPIVYYVGRKPKVEQLSNMIVRSCKCS

SEQ ID NO: 2 - protein, 442 aa, Homo sapiens
MHYCVLSAFLILHLVTVALSLSTCSTLDMDQFMRKRIEARIGQILSKLKITSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 3 - protein, 412 aa, Homo sapiens
MKMHLQRALVVLALLNFATVSLSLSTCTTLDFGHIKKKRVEAIRGQILSKLRITSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDERQDLGWKWVHEPKGYYANFCSGPCPYLRSADTTHSTVLGLYNTLNPEASASPCCVPQDLEPLTILYYVGRTPKVEQLSNMVVKSCKCS

(19) United States
(12) Patent Application Publication
(10) Pub. No.: US 2013/0344092 A1
(43) Pub. Date: Dec. 26, 2013

(54) Methods of Using Anti-Thymocyte Globulin and Related Agents

(71) Applicants: The Brigham and Women's Hospital, Inc., Boston, MA (US); Genzyme Corporation, Cambridge, MA (US)

(72) Inventors: Nader Najafian, Brookline, MA (US); Mohamed H. Sayegh, Westwood, MA (US); Melanie Ruzek, Danielson, CT (US); Srinivas Shankara, Shrewsbury, MA (US); John Williams, Hopkinton, MA (US); Johanne Kaplan, Sherborn, MA (US); John M. McPherson, Hopkinton, MA (US)

(73) Assignees: The Brigham and Women's Hospital, Inc., Boston, MA (US); Genzyme Corporation, Cambridge, MA (US)

(21) Appl. No.: 14/012,656

(22) Filed: Aug. 28, 2013

Related U.S. Application Data
(62) Division of application No. 12/302,598, filed on Oct. 2, 2009.
(60) Provisional application No. 60/803,575, filed on May 31, 2006.

(51) Int. Cl. A61K 35/14; A61K 39/395; C12N 5/0783

(52) U.S. Cl. CPC A61K 35/17; C12N 5/0637; A61K 39/39541
USPC 424/173.1; 424/93.71; 435/377

(57) ABSTRACT

Novel uses for anti-thymocyte globulin (ATG, e.g., Thymoglobulin®) and related compositions are described. In one aspect, ATG and, optionally, TGF-β are used for in vitro generation of regulatory T cells, which are useful for cell therapy of immune-mediated conditions. In another aspect, ATG is directly administered to a subject at a low dose (e.g., less than 1 mg/kg per day) to treat an immune-mediated condition. The immune-mediated conditions include, for example, transplant rejection, graft-versus-host disease, and autoimmune diseases.

This application claims priority to U.S. provisional application No. 60/803,575 filed on May 31, 2006, incorporated herein by reference in its entirety.

### STATEMENT OF RIGHTS

The U.S. Government may have certain rights in the present invention pursuant to funding of research under NIH/PPG Grant No. PO1 AI-050157.

### FIELD OF THE INVENTION

This invention relates to methods of treating immune-mediated diseases or conditions, such as transplant rejection, graft-versus-host disease, and autoimmune diseases. More specifically, the invention relates to the use of anti-thymocyte globulin (ATG) for ex vivo cell therapy treatment or for direct administration to patients.

### BACKGROUND OF THE INVENTION

Immune-mediated conditions such as transplant rejection, graft-versus-host disease, and autoimmune diseases are generally characterized by the presence of undesirable immune responses. Considerable advances have been made in the treatment of such conditions since the discovery of cyclosporine and other immunosuppressive drugs. For a review of current treatments for immune-mediated conditions, see, e.g., Paul W. E., Fundamental Immunobiology, 5^{th} ed. (2003) pp. 1621-1659, Immunotherapy. However, available immunosuppressive therapies may have limitations and significant adverse side effects, including the development of infections, cancer, and toxicity associated with long-term exposure to immunosuppressive drugs. Thus, the long-term transplant survival in a host continues to be a challenging problem.

Regulatory T cells (also known as “Tregs” or suppressor T cells) are specialized subsets of T lymphocytes that play important roles in maintaining immune system homeostasis by suppressing aberrant immune response (Fehervari et al., Curr. Opin. Immunol. 16:203-208 (2004) and Sakaguchi et al., Int. Rev. Immunol. 24:211-226 (2005)). A major type of Treg is characterized by the expression of CD4, the IL-2 receptor α-chain CD25, and the transcription factor FOXP3 (Sakaguchi, Clin. Invest. 112:1310-1312 (2003); Fontenot et al., Nat. Immunol. 4:330-336 (2003); and Hori et al., Science 299:1057-1061 (2003)). Normally, CD4⁺CD25⁺FOXP3⁺ T cells represent 4-5% of all circulating lymphocytes.

Emerging evidence in both rodents and humans suggests that CD4⁺CD25⁺Tregs are responsible for maintaining tolerance towards autoantigens (Sakaguchi et al., Int. Rev. Immunol. 24:211-226 (2005)) and alloantigens (Wood et al., Nat. Rev. Immunol. 3:199-210 (2003)). Tregs may also play a role in preventing human renal autoimmune diseases such as Goodpasture's disease (Salama et al., Kidney Int. 64:1685-1694 (2003)). It has been also reported that active regulation of the alloimmune responses by Tregs may function to maintain hyporesponsiveness to alloantigens in renal transplant patients (Najafian et al., J. Am. Soc. Nephrol. 13:252-259 (2002) and Salama et al., J. Am. Soc. Nephrol. 14:1643-1651 (2003)). In preclinical animal models, ex-vivo expanded Tregs were reported to protect mice from lethal GVHD (Taylor et al., Blood 99:3493-3499 (2002)). In fact, a clinical trial has been recently proposed to use ex-vivo expanded Tregs at the time of hematopoietic stem cell transplantation (Bluestone, Nat. Rev. Immunol. 5:343-349 (2005) and Gregori et al., Curr. Opin. Hematol. 12:451-456 (2005)).

Thus, a need exists to provide methods of generation and propagation of Tregs both in vivo and ex vivo to allow the development of novel therapeutic strategies for inducing immunologic tolerance in various immune-mediated conditions. This should allow minimization and possibly complete withdrawal of toxic immunosuppressive drugs.

### SUMMARY OF THE INVENTION

The present invention is based, in part, on the discovery and demonstration that culturing T lymphocytes with anti-thymocyte globulin (ATG) results in the generation of regulatory T cells that are functionally immunosuppressive. The invention is further based, in part, on the discovery and demonstration that ATG, such as Thymoglobulin® (Genzyme Corp.), promotes the generation of regulatory T cells in vitro in a dose-dependent manner at concentrations of 1-50 μg/ml, which are significantly lower than serum levels attained by dosages currently used in the clinic (˜100 μg/ml). Thus, ATG promotes expansion of regulatory T cells, and therefore ATG and ATG-like compositions may be used for (1) ex vivo expansion of these cells for subsequent cell therapy or (2) direct administration of ATG or ATG-like compositions to patients at appropriate lower dosages (than currently used) to expand and/or generate regulatory T cells in vivo. The methods of treatment are therefore aimed at suppressing aberrant immune responses, inducing tolerance, or otherwise normalizing the immune system homeostasis in the subject.

In one mode of therapy (“cell therapy”), T lymphocytes may be obtained from a mammal, propagated according to the methods of the invention in order to produce regulatory T cells, which are then administered to the mammal in need of the treatment. In such embodiments, the method of treating a mammal comprises administering to the mammal regulatory T cells made by methods of the invention.

In another embodiment, the cell therapy method includes a) expanding T lymphocytes obtained from a mammal in need of treatment according to the methods of the invention in order to produce regulatory T cells; b) depleting the circulating lymphocytes of the mammal; and c) administering to the mammal the regulatory T cells produced in step a). In some embodiments, the mammal's T cells are depleted by at least 10, 20, 50, 70, 80, 90, 95, 99%, or more, prior to receiving the expanded Tregs.

In another mode of therapy (“direct administration”), the invention provides a method of treating a mammal by administering ATG or an ATG-like composition directly to a mammal in need of the treatment, at a dose of less than 1 mg/kg/day, e.g., 0.01-0.5 mg/kg/day or 0.05-0.25 mg/kg/day. Preferred for administration to human subjects are human anti-human thymocyte versions of ATG, but other types of ATG, e.g., Thymoglobulin® (rabbit anti-human thymocyte globulin), may be used.

It is contemplated that the direct administration and cell therapy methods of the present invention may be combined. For example, ATG or an ATG-like composition is administered directly to a mammal in need of treatment, at a concentration of less than 1 mg/kg (e.g., 0.01-0.5 mg/kg/day or 0.05-0.25 mg/kg/day). Next, ex vivo expanded Tregs are administered to the mammal. Optionally, the two therapies may be administered at the same time, or in reverse order.

The mammal to be treated with the cell therapy or by the direct administration is preferably a human. The mammals to be treated include those having or at risk for immune-mediated conditions such as transplant rejection, graft-versus-host disease, autoimmune diseases and other immune conditions that are generally characterized by the presence of undesirable immune responses.

In another aspect, the invention provides a method of making regulatory T cells, comprising culturing T lymphocytes in the presence of an effective amount of ATG or an ATG-like composition for a period of time sufficient to generate regulatory T cells, for example, by conversion of a portion (e.g., at least 10%) of nonregulatory T cells (e.g., CD4⁺CD25⁻ cells) into regulatory T cells (e.g., CD4⁺CD25⁺), and/or (2) by expansion of pre-existing or the converted regulatory T cell population (e.g., CD4⁺CD25⁺ cells) by at least 30%.

In preferred embodiments, the ATG is anti-human thymocyte globulin, e.g., Thymoglobuin®.

The amount of ATG or the ATG-like composition and the period of time for culturing cells may vary. In some embodiments, the cells are incubated with ATG concentrations of 0.1 μg/ml to 1 mg/ml, preferably 1-100 μg/ml or 10-50 μg/ml, for a period of at least 8 hours, preferably for at least about 24 hours.

In further embodiments, in addition to being cultured with ATG or an ATG-like composition, the T lymphocytes are simultaneously or sequentially cultured with TGF-β and/or another agent that promotes regulatory T cells.

The T lymphocytes to be cultured are obtained from a mammal, preferably, from a human. For example, peripheral blood mononuclear cells (PMBCs), which contain T lymphocytes, can be isolated from the mammal's blood and then cultured according to the methods of the invention.

The invention further provides regulatory T cells made by the methods of the invention. In some embodiments, such cells are characterized by at least one or more of the following features:

(a) immunosuppressive activity in vitro and/or in vivo;

(b) expression of CD4, CD25, and FOXP3;

(c) expression of one or more of regulatory T cells markers (e.g., GITR, CTLA4, surface TGF-β, and CD103); and

(d) production of one or more Th2 cytokines (e.g., IL-4, IL-5, IL-10, IL-13, and INF-γ).

The foregoing summary and the following detailed description are exemplary and explanatory only and are not restrictive of the invention as claimed.

### BRIEF DESCRIPTION OF THE FIGURES

FIG. 1A shows results of a representative experiment, in which peripheral blood mononuclear cells (PBMCs) derived from healthy human volunteers were incubated with 10 μg/ml Thymoglobulin® for 24 hours or rabbit IgG (Rbt IgG) as a control. Cells were then harvested and analyzed by flow cytometry. The CD4⁺CD25⁺ T cell population increased significantly following a 24-hour treatment with Thymoglobulin®, but not with rabbit IgG. The ATG-induced CD4⁺CD25⁺ T cells expressed the regulatory T cells markers GITR, CTLA4, and FOXP3.

FIG. 1B shows the percent change in the CD4⁺CD25⁺ cell population as a function of time that the cells are incubated with ATG or rabbit IgG as a control (Rbt IgG). PBMCs derived from healthy human volunteers were incubated with 10 μg/ml Thymoglobulin® or rabbit IgG for 0, 6, 18, 24, 48, 72, or 96 hours. An increase in CD4⁺CD25⁺ T cell population was observed with an 18-hour and longer ATG incubation period.

FIG. 2 shows that a four day incubation of PBMCs with 100 μg/ml Thymoglobulin®, but not with rabbit IgG, resulted in an increase in the CD4⁺CD25⁺ T cell population. FOXP3 was expressed in at least 50% of the CD4⁺CD25⁺ T cell population induced by Thymoglobulin®.

FIG. 3A demonstrates that expansion of CD4⁺CD25⁺ T cells by ATG is accompanied by production of Th2 cytokines. PBMCs from healthy donors were incubated (in triplicates) in an ELISPOT plate with ATG, rabbit IgG, or medium alone (Roswell Park Memorial Institute (RPMI) medium) as controls for 48 hours. The quantification of spots (mean of at least three independent experiments for each cytokine) revealed an increase in INF-γ-, IL-4-, IL-5-, and IL-10-producing PBMCs incubated with ATG.

FIG. 3B demonstrates that neutralization of Th2 cytokines decreases expansion of regulatory T cells. Anti-IL-4, anti-IL-10, and anti-IL13 mAb or corresponding isotype controls were each added separately to PBMCs incubated with 10 μg/m ATG or rabbit IgG. Cells were then harvested after 24 hours and the percentage of CD4⁺CD25⁺FOXP3⁺ T cells, gated on CD4⁺ lymphocytes, was measured by flow cytometry. The neutralization led to significant decline in the percentage of CD4⁺ T cells expressing CD25 and FOXP3. (Mean values of two independent experiments are shown.)

FIG. 4A demonstrates that at various ratios to autologous responder cells, Thymoglobulin®-generated Tregs inhibited the activation of T cells stimulated with allogeneic dendritic cells.

FIG. 4B further shows that at various ratios to autologous responder cells, Thymoglobulin®-generated Tregs inhibited the activation of T cells stimulated with anti-CD3/anti-CD28 DynaBeads®.

FIG. 5 demonstrates that suppressor function of regulatory T cells generated by ATG is restricted to autologous responder cells. PBMCs were incubated with T cells previously incubated for 24 hours with Thymoglobulin® or rabbit IgG (labeled “Treg” and “Tcontrol”, respectively). The cells were then collected and washed twice with PBS and added into a mixed lymphocyte reaction (MLR) assay. After five days of incubation, the proliferative response was measured by ³H-thymidine incorporation. There was significant suppression of direct alloimmune response of autologous responders (Auto-R) to donor antigens (FIG. 5A) but not to third-party responder cells (Hetero-R) (FIG. 5B). The control did not exhibit any inhibition of MLR regardless of donor-recipient combination.

FIG. 6A demonstrates that ATG converts CD4⁺CD25⁻ into CD4⁺CD25⁺ T cells that express FOXP3. PBMCs were depleted of CD25⁺ cells using MACS columns. The cells were then incubated for 24 hours with ATG or rabbit IgG (control). Flow cytometric analysis showed that ATG induced an increase in CD25 expression on CD4⁺ T cells, which also showed high expression of FOXP3.

FIG. 6B shows that ATG induces proliferation of pre-existing CD4⁺CD25⁺ T cells. PBMCs were labeled with carboxyfluoroscein succinimidyl ester (CFSE) and cultured in the presence of mitogen phytohemagglutinin (PHA), 10 μg/ml ATG, or rabbit IgG for 72 hours. The proportion of proliferating CFSE-labeled cells was calculated. In the presence of ATG, CD4⁺CD25⁺ cells exhibited several discrete division cycles, while CD4⁺CD25⁻ cells exhibited only one division cycle. CD4⁺CD25⁺ cells from PBMCs incubated with rabbit IgG and the CD8⁺ cells did not proliferate. A representative experiment is shown.

FIG. 7 demonstrates that incubation of normal mouse splenocytes with anti-mouse thymocyte globulin (mATG) generates T cells that express markers of regulatory T cells. Mouse splenocytes were isolated and cultured with rabbit anti-murine thymocyte globulin (mATG) or control rabbit IgG. Four to five days later, cells were removed from culture and stained for markers of regulatory T cells (CD25, surface TGF-β, GITR, and CD103).

FIG. 8 demonstrates that the cells from mATG-stimulated cultures are able to inhibit ongoing immune responses in vitro. Normal mouse splenocytes were cultured with T-cell-activating polyclonal antibodies against CD3 and CD28 and in the presence of increasing concentrations of mATG-stimulated spleen cells or control rabbit IgG-stimulated cells. A dose-dependent inhibition of proliferative responses was observed in the presence of mATG-stimulated cells, but not with rabbit IgG-stimulated cells.

FIG. 9 demonstrates mATG-generated T cells are functionally immunosuppressive in vivo in a mouse acute graft-versus-host disease (GVHD) model. Cells from mATG-stimulated cultures were collected after five days in culture and injected intravenously into mice induced for a graft-versus-host reaction (allogenic spleen cell transfer). The transfer of mATG-stimulated spleen cells resulted in markedly reduced lethality from GVHD.

FIG. 10 demonstrates that ATG, but not rabbit Ig triggers significant expansion of regulatory T cells in PBMCs exposed to alloantigen (irradiated PBMCs, in a 1:1 ratio). PBMCs obtained from healthy volunteers (left panel, untreated) were cultured in the presence of alloantigen and 10 μg/ml of either ATG (Thymoglobuin®, top row) or rabbit Ig (bottom row). CD4⁺ cells were gated from both populations and subsequently examined for CD25 expression, as well as several regulatory T cell markers: GITR, CTLA4, and FOXP3. All Treg markers show increased expression in the ATG treatment, relative to the rabbit Ig treatment.

FIG. 11 demonstrates the importance of APCs in Treg generation in response to ATG (10 μg/ml Thymoglobuin®). Relative to a complete PBMC fraction (FIG. 11A; shown CD4⁺ gated), CD4⁺ cells enriched from PBMCs by negative selection fail to show expansion of CD4⁺CD25⁺FOXP3⁺ regulatory T cells, when cultured in the presence of ATG (FIG. 11B).

FIG. 12 demonstrates that allogenic APCs fail to promote the expansion of regulatory T cells in CD4⁺ cells. Negatively-selected CD4⁺ cells (FIG. 12A) cultured in the presence of APCs from allogenic PBMCs (in a 1:1 ratio) and ATG (10 μg/ml Thymoglobuin®), fail to show expansion of CD4⁺CD25⁺FOXP3⁺ regulatory T cells (FIG. 12B).

FIG. 13 demonstrates the role which monocytes (CD14⁺ cells) play in the expansion of regulatory T cells. Relative to a complete PBMC fraction (FIG. 13A), PBMCs depleted of monocytes (CD14⁺ cells) prior to incubation with ATG (10 μg/ml Thymoglobuin®), fail to show expansion of CD4⁺CD25⁺FOXP3⁺ cells (FIG. 13B).

### DETAILED DESCRIPTION OF THE INVENTION

The invention provides methods of making regulatory T cells, comprising culturing starting cells comprising T lymphocytes in the presence of an effective amount of ATG or an ATG-like composition to produce regulatory T cells. The invention further provides methods of treating immune-mediated conditions by, e.g., cell therapy with ATG-generated regulatory T cells or direct administration of ATG. For cell therapy, T lymphocytes may be obtained from a mammal and propagated according to the methods of the invention in order to produce regulatory T cells, which are then administered to the same mammal in need of the treatment. For direct administration, the invention provides methods of treating a mammal by administering ATG or an ATG-like compound directly to a mammal in need of the treatment, at a dose of less than 1 mg/kg per day. Both modes of treatment are described in detail below.

### ATG and ATG-Like Compositions

The methods of the invention involve novel uses of ATG and ATG-like compositions. The present invention is based, in part, on the realization that culturing T lymphocytes with ATG or an ATG-like composition will promote generation of functional regulatory T cells and, therefore, ATG and ATG-like compositions may be used for generation of these cells in vivo or in vitro.

Accordingly, in some embodiments, the methods of the invention comprise culturing a population of T cells in the presence of an effective amount of ATG or an ATG-like composition for a period of time sufficient to expand a regulatory T cell population. The regulatory cell population being expanded may originate from the pre-existing regulatory T cells and or nonregulatory T cells that are converted into regulatory T cells as a result of the culture with ATG or the ATG-like composition.

ATG is a globulin fraction of anti-serum raised against whole T cells (intact, lysed, or otherwise modified), typically, thymocytes or T cell lines. As used herein, the term “ATG” refers to the whole anti-serum, a globulin fraction thereof, or a subfraction of the globulin fraction that contains polyclonal anti-T lymphocyte antibodies.

The term “ATG-like composition” refers to a polyclonal antibody composition that is raised against a lymphocyte mixture and has the capacity to deplete peripheral T cells in the circulation, similarly to ATG. Examples of such compositions include anti-lymphocyte serum (ALS) and globulin (ALG) described in, e.g., Wood et al., Transplant. Proc. 3:676-679 (1971).

ATG is currently used for the treatment of various clinical conditions including prevention or rescue treatment of acute rejection in organ transplantation (Beiras-Fernandez et al., Exp. Clin. Transplant. 1:79-84 (2003)), conditioning for hematopoietic stem cell transplantation, treatment of severe aplastic anemia, various autoimmune diseases, and more recently for the treatment of graft-versus-host disease (GVHD) (Lowsky et al., N. Engl. J. Med. 353:1321-1331 (2005)). Commercial ATG products include, for example, Thymoglobulin® (Genzyme), Atgam™ (Pfizer), ATG-Fresenius™ S (Fresenius), and Tecelac™ (Biotest), any one of which can be used in the methods of the invention.

ATG binds to multiple cell surface proteins expressed on T cells (see, e.g., Bourdage et al., Transplantation 59:1194-1200 (1995); Bonnefoy-Bérnard et al., Transplantation 51:669-673 (1991)). The immunosuppressive activity of ATG has primarily been thought to result from the depletion of peripheral lymphocytes from the circulating pool through complement-dependent lysis or activation-associated apoptosis (Beiras-Fernandez et al., Exp. Clin. Transplant. 1:79-84 (2003); Genestier et al., Blood 91:2360-2368 (1998); Michallet et al., Transplantation 75:657-662 (2003); Zand et al., Transplantation 79:1507-1515 (2005)). Other potential mechanisms of action include modulation of surface adhesion molecules or chemokine receptor expression (Brennan, Transplantation 75:577-578 (2003)). Thymoglobulin® is approved in the United States for indications that include transplantation (1 mg/kg to 2.5 mg/kg per day for 2-14 days) and aplastic anemia (2.5 mg/kg to 3.5 mg/kg per day for 5 days). The currently used dosages lead to serum levels of the drug between 50-100 μg/ml (Lowsky et al., N. Engl. J. Med. 353:1321-1331 (2005); Zand et al., Transplantation 79:1507-1515 (2005)). These dosing regimens are based on ATG's efficacy to deplete T cells in the peripheral blood.

ATG can be produced by injecting isolated thymocytes from one species (e.g., human) into another species (e.g., rabbit or horse). Alternatively, ATG may be produced by injecting T cells of a specific cell line (e.g., Jurkat cells) into a host. For administration to humans, especially for long-term administration, fully or partially human forms of ATG may be preferred. Such forms of ATG may be obtained from transgenic animals that have been genetically engineered to express fully or partially human immunoglobulins. For example, human antibodies can be produced in transgenic animals, e.g., chickens, as described in PCT Publication WO 2003/081993 and U.S. Patent Application Publication No. 2005/246782. Such animals have disrupted endogenous immunoglobulin production and, when challenged with an antigen, produce human immunoglobulins encoded by engineered human DNA incorporated in the animal's DNA. In transgenic ayes, the human immunoglobulins can be recovered from the blood or eggs. As additional examples, methods for producing partially human antibodies in transgenic animals are described in, e.g., U.S. Patent Application Publication No. 2006/026696, PCT Publications WO 2005/007696, WO 01/19394, WO 2003/081992, WO 2003/097812 and WO 2004/044156.

ATG or an ATG-like composition may be used in two contexts in the present invention. In the first, ATG or an ATG-like composition is used at doses which expand Tregs by, e.g., conversion of non-regulatory T lymphocytes to Tregs, or by proliferation of existing Tregs. This use is applicable to both the cell therapy and direct administration methods. In the second context, ATG or an ATG-like composition is used in some embodiments of the cell therapy method as a lymphocyte depleting agent. ATG has been used extensively as a lymphocyte depleting agent and depletion regimens effective to this end would be well known to the skilled artisan.

### Regulatory T Cells

One of the goals of the present invention is to generate regulatory T cells. Regulatory T cells (also known as Tregs or suppressor T cells) are cells that are capable of inhibiting the proliferation and/or function of other lymphoid cells via contact-dependent or contact-independent (e.g., cytokine production) mechanisms. Types of regulatory T cells include (1) γεT cells, (2) Natural Killer T (NKT) cells, (3) CD8⁺ T cells, (4) CD4⁺ T cells and (5) double negative CD4⁻CD25⁻ T cells. See, e.g., Bach et al., Immunol. 3:189-98 (2003). For a detailed review of various types of regulatory T cells, see, e.g., Wing et al., Scand. J. Immunol. 62(1):1 (2005), Jonuleit et al., J. Immunol. 171:6323-6327 (2003), Horwitz et al., J. Leukocyte Biol. 74:471-478 (2003).

The so-called “naturally occurring” regulatory T cells are CD4⁺CD25⁺ T cells that express FOXP3. In addition to the FOXP3-expressing CD4⁺CD25⁺ cells, a minor population of CD8⁺FOXP3-expressing cells are also regulatory T cells. CD4⁺ T regs can be further divided into induced regulatory T cells that secrete interleukin-10 (IL-10) and TGF-β such as Tr1 cells and T-helper 3 (Th3) cells. Additional surface markers for CD4⁺CD25⁺ regulatory T cells include CD45RB, CD38, GITR, surface TGF-β, CTLA4, CD103, CD134, and CD62L.

The invention provides regulatory T cells made by the methods of the invention. The cells made by these methods are enriched in regulatory T cells (e.g., CD4⁺CD25⁺, more particularly, CD4⁺CD25⁺FOXP3⁺ cells, or another type of regulatory T cell as listed above) relative to the starting cells.

The starting cells as well as the resulting cells may contain cells of phenotypes other than regulatory T cells, such as, e.g., nonregulatory T cells, B cells, monocytes, granulocytes, erythrocytes, platelets, tolerogenic dendritic cells, etc. The T lymphocytes to be cultured are obtained from a mammal, e.g., mouse, rat, monkey, preferably human, especially if intended to be used for administration to humans. The starting cells, comprising T lymphocytes, are obtained from the whole blood or suitable lymphoid tissues (e.g., thymus, tonsils, lymph nodes, and spleen) of a mammal, and may contain at least 10%, 20%, 50%, 60%, 80%, 90% or more T lymphocytes as percent of all cells. In preferred embodiments, the starting cells are peripheral blood mononuclear cells (PBMCs), which is a fraction of the blood that contains T lymphocytes. PBMCs can be isolated, e.g., by conventional density gradient centrifugation (e.g., over Ficoll®-diatrizoate) as described in Coligan et al. (eds) Current Protocols of Immunology, John Wiley & Sons, Inc., 2006. The amount of a particular cell type can be determined using conventional clinical laboratory techniques (e.g., by flow cytometry as described in Robinson et al. (eds.) Current Protocols in Cytometry, John Wiley & Sons, Inc., 2006). Reference values for normal lymphocytes counts in normal human blood in humans are presented in Table 1. See also Feuci et al., Harrison's Principle of Internal Medicine, 14^{th} ed., McGraw Hill, 1998. The terms “T cell” and “T lymphocyte” are used interchangeably herein.

TABLE 1

| | Typical | Mean | Range | Mean | Range |
|---|---|---|---|---|---|
| Cell type | marker | (%) | (%) | (cells/μl) | (cells/μl) |
| Total T cells | CD3 | 71 | 55-87 | 1,586 | 781-2,391 |
| Total B cells | CD19 | 5 | 1-9 | 277 | 17-537 |
| Helper T cells | CD4 | 43 | 24-62 | 1,098 | 447-1,750 |
| Cytotoxic T cells | CD8 | 42 | 19-65 | 836 | 413-1,260 |

Prior to incubation with ATG or an ATG-like composition, the starting cells may be optionally enriched in a certain type of T lymphocytes by, e.g., cell sorting. For example, the starting cells may be enriched in CD4⁺ T cells to contain up to 30%, 40% 50%, 60%, 70%, or 80% of such cells as percent of all starting cells. The starting cells may be optionally enriched in CD4⁺CD25⁺ T cells to contain up to 30%, 40% 50%, 60%, 70%, or 80% of such cells as percent of all starting cells and/or CD4⁺CD25⁻ T cells to contain up to 1%, 2%, 3%, 4%, 5%, 10%, 20%, 50%, 60%, 70%, or 80% of such cells as percent of all starting cells. The enrichments can be performed using conventional cell sorting techniques.

In certain embodiments, the starting cells are incubated with ATG or an ATG-like composition for a period of time sufficient to (1) convert a portion of nonregulatory T cells into regulatory T cells, and/or (2) to result in expansion of a regulatory T cell population.

In preferred embodiments, ATG is anti-human thymocyte globulin, e.g., Thymoglobuin®. In other embodiments, ATG is, for example, Atgam™, ATG-Fresenius™ S, and Tecelac™.

The amount of ATG or an ATG-like composition and/or the period of time for culturing the cells may vary. In some embodiments, the starting cells are incubated with ATG, such as, e.g., Thymoglobulin®, at an effective concentration from 0.1 μg/ml to 1 mg/ml, from 0.5 μg/ml to 500 μg/ml, preferably 1-100 μg/ml, more preferably 1-50 μg/ml, for example, 10-50 μg/ml, 1-40 μg/ml, 1-30 μg/ml, 1-20 mg/ml, 5-30 μg/ml, 5-40 μg/ml, and 10-30 μg/ml. If both ATG and an ATG-like composition are used together, when calculating the effective concentration, their concentrations may need to be added, or otherwise adjusted to arrive at the effective concentration.

The period of incubation with ATG and/or ATG-like composition may be at least 8, 12, 18, 24, 36, 48, 60, 72, 84, or 96 hours, for example, for 8-96, 12-48, 18-36, or at least 24 hours. It may also be desirable to repeat the incubation cycles two or more times over several weeks in order to obtain adequate cell numbers. In illustrative embodiments, PMBCs are incubated with 1-100 μg/ml Thymoglobulin® from 8 to 96 hours, optimally, with about 10 μg/ml for about 24 hours. Other conditions of cells cultures will be readily determined by a skilled artisan. See, e.g., Davis (ed.) Basic Cell Culture, 2^{nd} ed., 2002.

In further embodiments, in addition to being cultured with ATG or an ATG-like composition, the T lymphocytes are simultaneously or sequentially cultured with TGF-β and/or another agent that promotes regulatory T cells, as described below.

In some embodiments, at least 10%, 20%, 30%, 40%, 50% or more of nonregulatory T cells in the starting cells, e.g., CD4⁺CD25⁻, are converted to regulatory T cells as a result of culturing the cells with ATG or an ATG-like composition. In addition, or alternatively, the incubation with ATG or an ATG-like composition may result in expansion of the starting regulatory T cell population by at least 30%, 50%, 80%, 100%, 200%, 300% or more. In some embodiments, CD4⁺CD25⁺ T cells proliferate at an average rate of one or more divisions every 96, 72, 48, 36, 24 hours or a shorter time period. Such expansion may be due to the proliferation of pre-existing regulatory T cells or due to the conversion of at least a portion of nonregulatory cells (e.g., CD4⁺CD25⁻) to regulatory T cells (e.g., CD4⁺CD25⁺).

In some embodiments, the regulatory T cells made by the methods of the invention are characterized by at least one or more of the following properties:

(a) immunosuppressive activity in vitro and/or in vivo;

(b) expression of CD4, CD25 and FOXP3 (e.g., at least 30%, 50%, 70% or 90% of CD4⁺CD25⁺ cells also express FOXP3);

(c) expression of one or more of regulatory T cells markers (e.g., GITR, CTLA4, surface TGF-β, CD103, etc.); and

(d) production of one or more Th2 cytokines (e.g., IL-4, IL-5, IL-10, IL-13 and INF-γ).

Assays for determining the above properties are well known. See, e.g., Paul W. E., Fundamental Immunobiology, 5^{th} ed. (2003) and the Examples. Some of the more frequently used in assays are as follows:

1) flow cytometry analysis, wherein co-expression of CD4, CD25, and/or FOXP3 and/or CD62L and/or GITR and/or CTLA4 and/or surface TGF-β and/or CD103 and/or CD134 is used as indication of a regulatory T cell phenotype (Jonuleit et al., J. Immunol. 171:6323-6327 (2003);

2) inhibition of T cell proliferation in a co-culture system as described in, e.g., Chen et al., J. Exp. Med. 198:1875-1886 (2003) or in the Examples. In this assay, regulatory T cells are added to responder T cells and the co-culture is stimulated with anti-CD3 or allogeneic lymphocytes. In the presence of regulatory T cells, the responder T cells become unable to proliferate in response to these stimuli. The degree of proliferation is typically measured by tritiated thymidine incorporation; and

3) cytokine profiling as described in, e.g., Barrat et al., J. Exp. Med. 195:603-616 (2002); Jonuleit et al., J. Immunol. 171:6323-6327 (2003). In this assay, a supernatant from cultured regulatory T cells is analyzed for the presence of the immunosuppressive cytokines such as, e.g., IL-10 and TGF-β, known to be produced by regulatory T cells.

### TGF-β and Other Agents

In further embodiments, in addition to being cultured with ATG or an ATG-like composition, the T lymphocytes are simultaneously or sequentially cultured with TGF-β and/or another agent that promotes regulatory T cells. For example, in some embodiments, the methods comprise culturing a population of T cells simultaneously in the presence of (1) an effective amount of ATG or an ATG-like composition and (2) TGF-β for a period of time sufficient to expand a regulatory T cell population. In other embodiments, the methods comprise sequentially (1) culturing a population of T cells in the presence of an effective amount of ATG or an ATG-like composition and then (2) culturing these cells in the presence of an effective amount of TGF-β for a period of time sufficient to expand a regulatory T cell population. In other embodiments, the methods comprise (1) culturing a population of T cells in the presence of an effective amount of TGF-β and then (2) culturing these cells in the presence of an effective amount of ATG or an ATG-like composition for a period of time sufficient to expand a regulatory T cell population. In addition to the incubation with ATG or an ATG-like composition, the methods of the invention may include, among other manipulations, incubating the lymphocytes isolated from a mammal with TGF-β and re-administering the lymphocytes to the mammal as described in, e.g., U.S. Pat. No. 6,759,035.

In the methods of the invention, TGF-β may be naturally occurring or engineered, e.g., as described below. In some embodiments, TGF-β is active, e.g., mature TGF-β. In some embodiments, TGF-β is TGF-β1, TGF-β2, or TGF-β3. The appropriate effective amounts of TGF-β may range from about 10 μg to about 10 ng/ml, e.g., 0.1-5 ng/ml or about 1 ng/ml.

TGF-β is naturally secreted in either a so-called “small latent complex” (100 kDa) in which the biologically active TGF-β is noncovalently associated with its pro domain (“latency-associated peptide,” LAP) and in a so-called “large latent complex” (220 kDa) additionally containing latent TGF-β biding protein (LTBP). The latent forms are unable to bind to TGF-β receptors until active, i.e., mature, TGF-β is released from the complex. For a more detailed review of the latent forms and activation process, see, e.g., Cytokine Reference, eds. Oppenheim et al., Academic Press, San Diego, Calif., 2001, pp. 724-725. In cell-based expression systems, TGF-β can be engineered to be expressed in its mature form and its biological activity can be recovered, e.g., by disulfide exchange. There are three known mammalian isoforms of TGF-β (TGF-β1 to TGF-β3), all of which are homologous among each other (60-80% identity). A partial listing of protein accession number for the three mammalian isoforms is provided in Table 2.

TABLE 2

| Species | TGF-β1 | TGF-β2 | TGF-β3 |
|---|---|---|---|
| Human | PO1137 | PO8112 | P109600 |
| Mouse | P04202 | P27090 | P171125 |
| Rat | AAD20222 | AAD24484 | Q07258 |
| Porcine | AAA616 | AAB03850 | P15203 |
| Simian | P09533 | WFMKB2 | |

The structural and functional aspects of TGF-β as well as TGF-β receptors are well known. See, e.g., Oppenheim et al. (eds) Cytokine Reference, Academic Press, San Diego, Calif., 2001. Thus, for the purposes of the present disclosure, the term “TGF-β” refers not only to the naturally occurring forms but also to engineered TGF-β that retain the ability to bind to one or more TGF-β receptors (TβRI, TβRII, or TβRIII). Engineered TGF-β may contain only a partial or a mutated amino acid sequence of the naturally occurring TGF-β. For example, engineered TGF-β may contain native sequences in which conservative substitutions were made and/or nonessential amino acids were deleted. For example, engineered TGF-β may comprise a sequence, which is at least 80%, 85%, 90%, 95%, 96%, 97%, 98%, 99%, or 100% identical to the 112 amino acid C-terminal portion of any one of SEQ ID NO: 1, 2, or 3 over the entire length of this C-terminal portion.

In addition to ATG or ATG-like compositions and, optionally, TGF-β, the cells may be cultured, simultaneously or sequentially, in the presence of an effective amount of another agent(s) that promote regulatory T cells, such as, e.g., (1) IL-10, (2) IL-10 and IL-4, (3) IL-10 and IFN-α, (4) vitamin D3 and dexamethasone, (5) vitamin D3 and mycophenolate mofetil, and (6) rapamycin. (See, e.g., Barrat et al., J. Exp. Med. 195:603-616 (2002); Jonuleit et al., J. Immunol. 171:6323-6327 (2003); Gregori et al. J Immunol. 167:1945-1953 (2001); Battaglia et al., Blood 105:4743-4748 (2005)).

### Therapeutic Uses

The therapeutic methods of invention provide at least two modes of therapy: cell therapy and direct administration. In cell therapy, T lymphocytes may be obtained from a mammal, propagated according the methods of the invention in order to produce regulatory T cells, which are then administered to the mammal in need of the treatment. Thus, this method of treating a mammal comprises administering to the mammal regulatory T cells made by the method of the invention.

In some embodiments, the method of cell therapy comprises obtaining T cells (e.g., in the form of PBMCs) from a mammal, culturing the cells with ATG or an ATG-like composition and, optionally with TGF-β or another agent that promotes regulatory T cells, thereby generating a population of regulatory T cells, and then administering the regulatory T cells to the mammal. The administration of cells to a recipient may be accomplished by a variety of routes, e.g., by administration directly to a tissue or organ of interest or by intravascular administration, including intravenous or intraarterial administration, intraperitoneal administration, etc. The cells can be infused by intravenous (i.v.) administration over a period of time, from several minutes to several hours. Additional agents such as buffers or preservants may be added to the cells. After the administration of the cells into the patient, the effect of the treatment may be evaluated and additional rounds of therapy may be performed, if needed.

In some embodiments, the Tregs may be obtained from a fraction of PBMCs. Preferably, that fraction comprises autologous monocytes or dendritic cells. Optionally, B cells may be absent.

In another embodiment, the cell therapy method includes a) expanding T lymphocytes obtained from a mammal in need of treatment according to the methods of the invention in order to produce regulatory T cells; b) depleting the circulating lymphocytes of the mammal; and c) administering to the mammal the regulatory T cells produced in step a). In some embodiments, the mammal's T cells are depleted by at least 10, 20, 50, 70, 80, 90, 95, 99%, or more, prior to receiving the expanded Tregs.

The direct administration mode of therapy involves treating a mammal by administering ATG or an ATG-like composition directly to a mammal in need of the treatment, at a dose of less than 1, 0.9, 0.8, 0.7, 0.6, 0.5, 0.4, 0.3, 0.2, or 0.1 mg/kg/day, e.g., 0.01-0.5 mg/kg/day or 0.05-0.25 mg/kg/day. It is theorized, but is not relied on for the purposes of the invention, that such lower doses may not necessarily result in complete T lymphocyte depletion, but nevertheless would be sufficient to stimulate generation of regulatory T cells a in subject. As reported by Guttmann et al., Transplant. Proc. 29 (Suppl. 7A):24S-26S (1997), after an intravenous dose of 1.25 to 1.5 mg/kg/day (over 4 hours for 7-11 days) 4-8 hours post-infusion, Thymoglobulin® serum levels are on average 21.5 μg/ml (10-40 μg/ml) with a half-life of 2-3 days after the first dose, and 87 μg/ml (23-170 μg/ml) after the last dose. Therefore, the effective dosages employed for “direct administration” are expected to result in lower serum concentrations of ATG or an ATG-like composition than those cited by Guttmann. Accordingly, in some embodiments of direct administration, treatment regimens are expected to result in maximal serum concentrations of ATG, or an ATG-like composition, of less than 15, 10, 5, 1, 0.5, or 0.1 μg/ml, which are expected to be efficacious.

The treatment may be performed over the course of several days to several weeks. In some embodiments, ATG or an ATG-like composition is administered repeatedly. For example, ATG or an ATG-like composition may be administered to the subject, at a dose indicated above, daily or every other day, or less frequently, for 5 to 10 days or two to three weeks, or two months, or longer. It may also be desirable to repeat the treatment cycle two or more times as necessary to achieve a desired effect.

Preferred for administration to human subjects are human anti-human thymocyte versions of ATG, but other types of ATG, e.g., Thymoglobulin®, may be used. The preferred method of administration is intravenous infusion over a period of time. For general methods of administration for ATG, see, e.g., Physicians' Desk Reference (PDR®) 2005, 59^{th} ed., Medical Economics Company, 2004; and Remington: The Science and Practice of Pharmacy, eds. Gennado et al., 21th ed., Lippincott, Williams & Wilkins, 2005).

It is further contemplated that the treatment methods of the present invention may be combined. For example, ATG or an ATG-like composition is administered directly to a mammal in need of treatment, at a concentration of less than 1 mg/kg (e.g., 0.01-0.5 mg/kg/day or 0.05-0.25 mg/kg/day). Next, ex vivo expanded Tregs are administered to the mammal. Optionally, the two therapies may be administered at the same time, or in reverse order.

Examples of mammals to be treated with cell therapy or direct administration treatment regimens of the invention include humans or other primates (e.g., chimpanzees), rodents (e.g., mice, rats, or guinea pigs), rabbits, cats, dogs, horses, cows, and pigs. Effective dosages achieved in one animal may be converted for use in another animal, including humans, using conversion factors known in the art. See, e.g., Freireich et al., Cancer Chemother. Reports 50(4):219-244 (1966) and Table 3 for equivalent surface area dosage factors). Examples of autoimmune disease and transplantation models and appropriate methods can be found in the Examples and are known in the art (see, e.g., Cohen et al. (eds.), Autoimmune Disease Models, Academic Press, 2005).

TABLE 3

| | From: | | | | |
|---|---|---|---|---|---|
| | Mouse | Rat | Monkey | Dog | Human |
| To: | (20 g) | (150 g) | (3.5 kg) | (8 kg) | (60 kg) |
| Mouse | 1 | 0.5 | 0.25 | 0.17 | 0.08 |
| Rat | 2 | 1 | 0.5 | 0.25 | 0.14 |
| Monkey | 4 | 2 | 1 | 0.6 | 0.33 |
| Dog | 6 | 4 | 1.7 | 1 | 0.5 |
| Human | 12 | 7 | 3 | 2 | 1 |

The mammals to be treated include those having, or at risk for, immune-mediated conditions such as transplant rejection (including acute and chronic transplant rejection and corticosteroid-resistant rejection), graft-versus-host disease, autoimmune diseases and other immune conditions that are generally characterized by the presence of undesirable immune responses.

In case of organ (e.g., kidney) or tissue (e.g., bone marrow) transplantation, the mammal may receive treatment by cell therapy and/or direct administration prior to and/or following the transplantation. Cell therapy and direct administration treatment regimens of the invention may also be combined with other immunosuppressive therapies, e.g., cyclosporine.

The methods of the invention can be used to treat a mammal that has an autoimmune disease such, e.g., systemic lupus erythematosus (SLE) and autoimmune rheumatoid arthritis (RA).

Example of additional autoimmune diseases include insulin-dependent diabetes mellitus (IDDM; type I diabetes), inflammatory bowel disease (IBD), graft-versus-host disease (GVHD), celiac disease, autoimmune thyroid disease, Sjögren's syndrome, Goodpasture's disease, autoimmune gastritis, autoimmune hepatitis, cutaneous autoimmune diseases, autoimmune dilated cardiomyopathy, multiple sclerosis (MS), myasthenia gravis (MG), vasculitis (e.g., Takayasu's arteritis and Wegener's granulomatosis), autoimmune diseases of the muscle, autoimmune diseases of the testis, autoimmune ovarian disease, autoimmune uveitis, Graves' disease, psoriasis, ankylosing spondylitis, Addison disease, Hashimoto thyroiditis, idiopathic thrombocytopenic purpura, and vitiligo.

The methods of the invention are expected to slow the progression of autoimmune disease, improve at least some symptoms or asymptomatic pathologic conditions associated with a disease, and/or increase survival. For example, the methods of the invention may result in a reduction in the levels of autoantibodies, B cells producing autoantibodies, and/or autoreactive T cells. The reduction in any of these parameters can be, for example, at least 10%, 20%, 30%, 50%, 70% or more as compared to pretreatment levels. With regard to organ and tissue transplantation, survival of the graft is expected to be prolonged by at least 50%.

The invention further provides methods of preserving or improving kidney function in a mammal with an autoimmune disease that compromises kidney function. Examples of autoimmune diseases that may compromise kidney function include SLE (e.g., lupus nephritis), Goodpasture's disease, Wegener's granulomatosis (Wegener's syndrome), Berger's disease (IgA nephropathy), and IgM nephropathy. In some of the patients afflicted with such diseases, the treatment is expected to result in improvement of kidney function (e.g., slowing the loss of, preserving, or improving the same) as indicated by, e.g., a change in systemic blood pressure, proteinuria, albuminuria, glomerular filtration rate, and/or renal blood flow.

### Lymphocyte Depletion

One embodiment of the cell therapy method of treatment involves treating a mammal having, or at risk for, immune-mediated conditions or diseases, comprising the steps of:

(a) expanding T lymphocytes obtained from a mammal in need of treatment according to the methods of the invention in order to produce regulatory T cells; and

(b) depleting the circulating lymphocytes of the mammal; and

(c) administering the regulatory T cells generated in step a) to the mammal.

Depletion of circulating lymphocytes can be accomplished by administering a lymphocyte-depleting agent to the mammal or otherwise exposing the mammal to conditions that result in a loss of a substantial fraction of lymphoid cells (e.g., lymphocytes, natural killer (NK) cells, monocytes, and/or dendritic cells, etc.) in the mammal. Lymphocytes to be depleted may be T lymphocytes (T cells) and/or T and B lymphocytes. In the depletion phase, T cell counts are reduced by at least 30%, 40%, 50%, 60%, 70%, 80%, 90%, 95%, or more, and optionally, B lymphocyte (B cell) counts are reduced by at least 30%, 40, 50%, 60%, 70%, 80%, 90%, 95%, or more. In preferred embodiments, the depleted lymphocytes are predominantly T cells, which means that the percentage of depleted T cells is greater (e.g., 1.2-, 1.5-, 2-, 5-, 10-fold, or more) than the percentage of depleted B cells.

The level of lymphocyte depletion can be readily assessed by, for example, measuring the amount of peripheral blood lymphocytes (PBLs). Lymphocyte counts can be determined using conventional clinical laboratory techniques (e.g., by flow cytometry). Reference values for normal PBL levels in humans are presented in Table 4.

TABLE 4

| | Typical | | | Mean | Range |
|---|---|---|---|---|---|
| Cell Type | Marker | Mean (%) | Range (%) | (cells/μl) | (cells/μl) |
| Total T cells | CD3 | 71 | 55-87 | 1,586 | 781-2,391 |
| Total B cells | CD19 | 5 | 1-9 | 277 | 17-537 |
| Helper T cells | CD4 | 43 | 24-62 | 1,098 | 447-1,750 |
| Cytotoxic | CD8 | 42 | 19-65 | 836 | 413-1,260 |
| cells | | | | | |

In some embodiments, the lymphocyte-depleting agent is an anti-lymphocyte antibody, e.g., anti-T cell antibodies, e.g., anti-thymocyte globulin (ATG), such as, e.g., Thymoglobulin®, Atgam™, Fresenius™, and Tecelac™. ATG is a polyclonal antibody directed against thymocytes. Currently marketed ATG products are produced by injecting thymocytes from one species (e.g., human) into another species (e.g., rabbit or horse). ATG binds to cell surface proteins such as lymphocyte surface antigens CD2, CD3, CD4, CD8, CD11a, CD18, CD25, HLA DR, and HLA class I (Bourdage et al., Transplantation 59:1194-1200 (1995)). ATG is believed to induce immunosuppression primarily as a result of T cell depletion (see, e.g., Bonnefoy-Bérnard et al., Transplantation 51:669-673 (1991)) and has been previously used for pretreating transplant patients to reduce the risk of rejection in the context of organ transplantation.

In addition to ATG, the lymphocyte-depleting agent consists of or comprises a monoclonal or polyclonal antibody directed to one or more specific lymphocyte surface antigens, e.g., anti-CD52 antibody (e.g., Campath®), anti-CD3 antibody (e.g., OKT3®), anti-CD4 antibody (OKT™), anti-CD25 (IL-2R) antibody (e.g., daclizumab), anti-CD5 antibody, anti-CD7 antibody, anti-TCR antibody, anti-CD2 (e.g., Siplizumab™), or an antibody against any of other lymphocyte surface antigens specified above, etc.

In some embodiments, the lymphocyte-depleting agent is a corticosteroid.

In some embodiments, conditions that result in depletion of lymphocytes include exposure to gamma radiation.

A combination of any suitable agents and/or conditions to deplete lymphocytes can be also used.

The following Examples are provided for illustrative purposes and are not intended to be limiting.

### EXAMPLES

### Example 1

### ATG Expands CD4⁺CD25⁺ Regulatory T Cells

Blood from ten healthy donors was obtained in heparinized tubes, and peripheral blood mononuclear cells (PBMCs) were isolated by standard Ficoll® density gradient centrifugation, PBMCs were incubated at 37° C., 5% CO₂, with 10 μg/ml Thymoglobulin® or rabbit IgG (control) for varying time periods of 0, 6, 18, 24, 48, 72, and 96 hours. These cultures are referred to herein as “generating cultures.”

Cells were then harvested and analyzed using flow cytometric analysis. 2×10⁵ cells per sample were stained with anti-human CD4-allophycocyanin (APC), CD25-phycoerythrin (PE), glucocorticoid-induced tumor necrosis factor receptor (GITR)-flourescein isothiocyanate (FITC), and CD8-APC (BD Bioscience, San Jose, Calif.; eBioscience, San Diego, Calif.). For the intracellular CTLA-4 staining, cells were permeabilized with Perm buffer (BD Biosciences, San Jose, Calif.) for 20 minutes at 4° C. and labeled with anti-CTLA-4 for 30 minutes at 4° C. For flow cytometric analysis of forkhead box P3 (FOXP3), 1×10⁶ cells were first stained with anti-human CD4-APC and CD25-PE. After washing, cells were re-suspended in 1 ml of cold Fix/Perm buffer (eBioscience, San Diego, Calif.) and incubated at 4° C. overnight in the dark. After a wash with 2 ml of Permeabilization buffer, cells were blocked with 2% normal rat serum for fifteen minutes. Anti-human FOXP3-FITC antibody (PCH101, eBioscience) was then added, and cells incubated at 4° C. for another 30 minutes in the dark. Finally, cells were washed with 2 ml of Permeabilization buffer and analyzed by flow cytometry using a FACSCalibur™ flow cytometer and CellQuest™ software. Student's t-test was used for comparison of means between experimental groups. Differences that had p values of less than 0.05 were considered statistically significant.

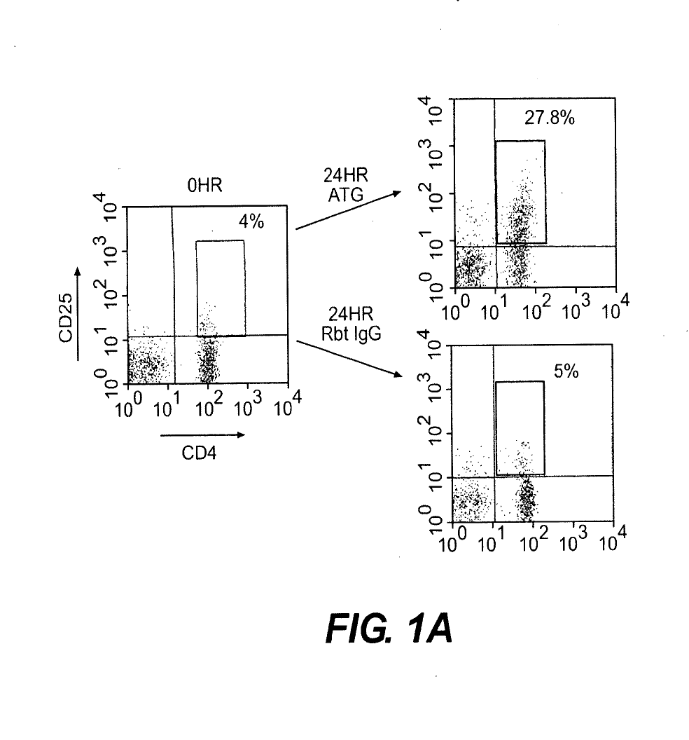

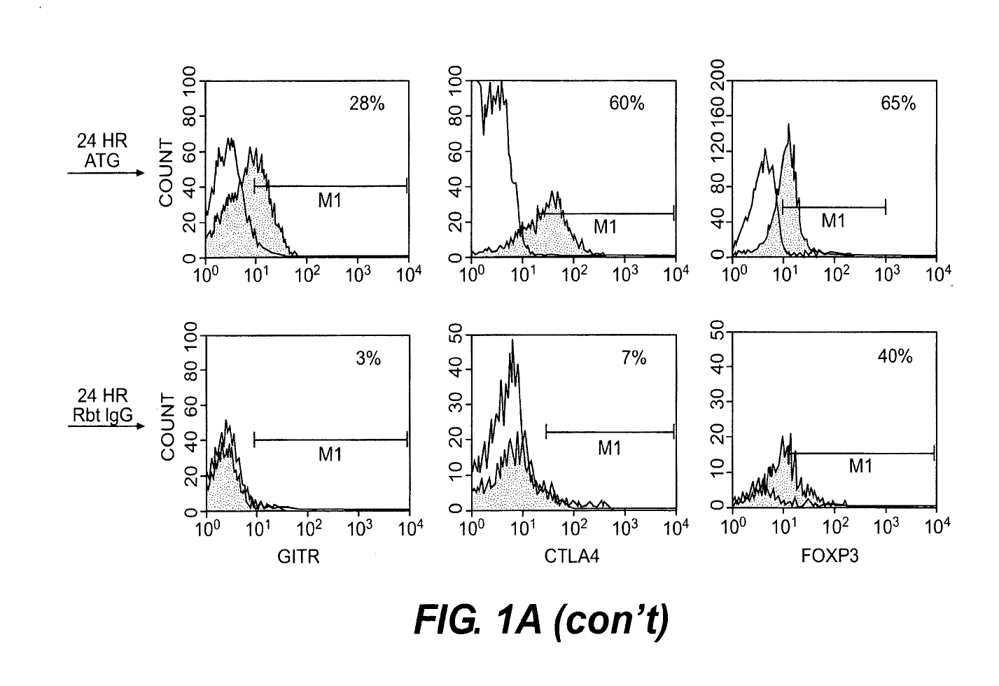

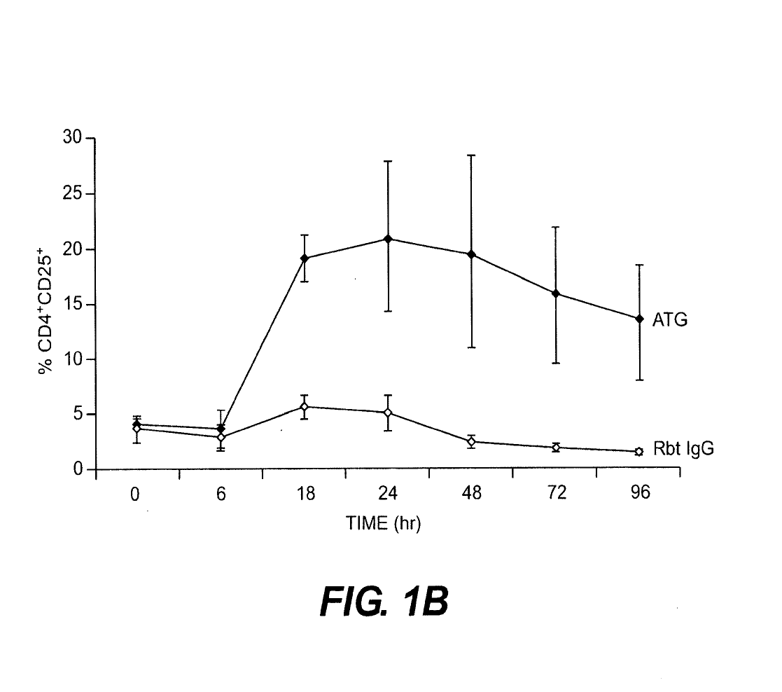

Significant upregulation of CD25 expression was observed after an 18-hour or longer incubations with ATG, with a maximal expression achieved with a 24-hour incubation (see FIG. 1B). A representative experiment demonstrating enrichment of the CD4⁺CD25⁺ T cell population at 24 hours (peak expression, 20.5±7.8% vs. 4.5±1.6%, p=0.002, n=7) is shown in FIG. 1A.

The regulatory function in humans is thought to be mainly attributed to the CD25^{high} subset of CD4⁺ cells (Baecher-Allan et al., J. Immunol. 167:1245-1253 (2001)). Thus, the frequency of CD4⁺CD25^{high} subpopulation in Thymoglobulin®-treated cells was evaluated. It was found that this subpopulation was significantly increased in Thymoglobulin®-treated group vs. rabbit IgG controls (6.5±2.9% vs. 0.7±0.5% of CD4⁺CD25⁺ cells, p=0.001, n=8). Similar results were obtained with the ATG from Fresenius following a 24-hour incubation at 10 μg/ml: CD4⁺CD25⁺ T cells (16.7±4.2% vs. 4.5±1.6%, p=0.04, n=3) and the CD25^{high} subset (4.7±1 vs. 0.7±0.5°/h, p=0.02, n=3).

CD4⁺CD25⁺ T cells incubated with Thymoglobulin® had significantly higher expression of regulatory T cell markers GITR (32±12% vs. 6.6±4%, n=5), intracellular CTLA4 (41.3±19.5% vs. 7±1.8%, n=4) and FOXP3 (65.3±21.5 vs. 43.8±12.3, n=5) as compared to rabbit IgG controls (FIG. 1A). CD4⁺CD25⁺FOXP3⁻ cells as percent of CD4⁺ T cells were significantly higher in the Thymoglobulin®-expanded cells relative to rabbit IgG controls (10.4±2.5% vs. 2.2±0.5%, p<0.0001, n=8). Expression of all three regulatory markers was even more enhanced in the CD4⁺CD25^{high} population after incubation with ATG (GITR: 49.4±15.9%, n=7; CTLA-4: 55±24.4%, n=6; FOXP3: 71±14.7%, n=5).

Prior work indicated that FOXP3 expression can be induced in CD4⁺CD25⁻ T cells, thereupon these cells become able to perform regulatory functions (Zheng et al., J. Immunol. 172:5213-5221 (2004)). To explore this possibility, CD4⁺CD25⁻ T cells were selected and incubated with ATG. The cells showed only minimal increase in GITR (5.6±4.4% vs. 0) and CTLA-4 (11.5±4.2% vs. 0). Furthermore, the percentage of CD4⁺CD25⁻FOXP3⁺ T cells (gated on CD4⁺ T cells) was minimal after incubation with ATG vs. rabbit IgG (1.1±0.6 vs. 0.7±0.4). The results indicate that ATG did not induce CD4⁺CD25⁻FOXP3⁺ T cells. In addition, no CD8⁺FOXP3⁺ T cells were detected upon ATG treatment. Overall, there was a slight decrease in the CD8⁺ T cells upon incubation with ATG as compared to rabbit IgG control (21.96±4.5% vs. 25.6±5.1%, n=8, p=0.02), but there was no significant difference in the percentage of CD8⁺CD25⁻ T cells between ATG-treated cells and control (11.3±5.6% vs. 14.9±6%, n=5).

### Example 2

### ATG Generates CD4⁺CD25⁺FOXP3⁺ Regulatory T Cells

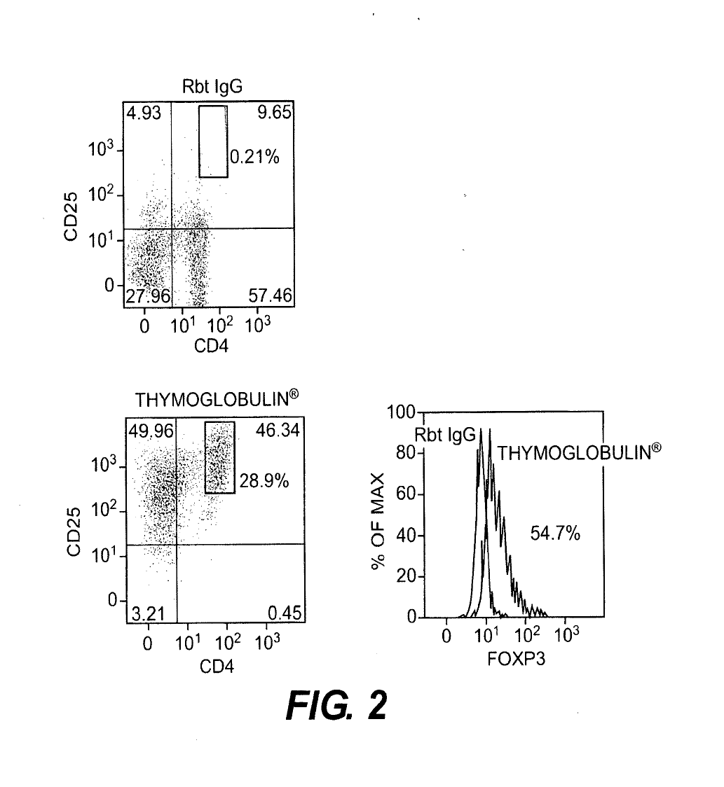

Human PBMCs were placed into culture for 5-7 days in AIM V media containing non-heat inactivated 10% human AB serum. Cultures were supplemented with Thymoglobulin® at 100 μg/ml or rabbit Ig (control). Expression of cell surface receptors was determined by flow cytometry. Cells were washed in PBS and resuspended in PBS supplemented with 1% human AB serum. Fluorescently labeled anti-CD4 and anti-CD25 antibodies were added to the cells and incubated for 30 minutes at 4° C. in the dark. Cells were washed then incubated in Fix/Perm buffer (eBioscience) at 4²C for 30 minutes. Cells were then washed and stained for 30 minutes with anti-human FOXP3 antibody (eBioscience). Cells were washed, resuspended in PBS and analyzed on a FacsCalibur cytometer (BD Biosciences). FIG. 2 illustrates that in comparison to rabbit IgG control, a four day treatment with Thymoglobulin® of PBMCs generated a significant population of CD4⁺CD25⁺ cells, more than half of which are FOXP3 positive.

### Example 3

### ATG Expands Regulatory T Cells in a Dose-Dependent Manner

PBMCs were isolated and incubated for 24 hours with 1, 5, 10, 50 and 100 μg/ml ATG or a rabbit IgG as per Example 1. Cells were then harvested and analyzed by flow cytometry.

As shown in Table 5, a dose-dependent increase in percentage of CD4⁺CD25⁺ T cells was observed in between 1 and 10 μg/ml of ATG. At higher concentrations, no appreciable further increase was observed. Increased activation of CD4⁺ T cells was also observed as indicated by the percentage of CD4⁺CD69⁺ cells (see Table 5). FOXP3 expression in CD4⁺CD25⁺ T cells remained substantially the same with increasing the dose of ATG (13±4.2% at 50 μg/ml to 15.2±0.35% at 100 μg/ml).

TABLE 5

| ATG | % CD4⁺CD25⁺ | % CD4⁺CD69⁺ |
|---|---|---|
| (μg/ml) | T cells* | T cells** |
| 1 | 6.3 ± 0.5 | 4.4 ± 4.8 |
| 5 | 12 ± 4.9 | 15.6 ± 7.7 |
| 10 | 20 ± 6.5 | 12 ± 7.2 |
| 50 | 21.7 ± 5 | 21 ± 5.6 |
| 100 | 21 ± 6.7 | 22 ± 5 |

*Rabbit IgG controls were 3-5%;
**Rabbit IgG controls were less than 1%.

### Example 4

### Role of Cytokines in the Expansion of Regulatory T Cells by ATG

To assess the role of cytokines in the expansion of regulatory T cells, the frequency of cytokine-producing cells (IL-4, IL-5, IL-10, IL-13, and INF-γ) was measured by the ELISPOT assay as previously described (Najafian et al., J. Am. Soc. Nephrol. 13:252-259 (2002)). PBMCs isolated from healthy volunteers either with ATG or rabbit IgG control in ELISPOT plates for 48 hours. Cells were tested in triplicate wells. The resulting spots were counted on a computer assisted ELISASpot Image Analyzer (Cellular Technology Limited). The frequencies were expressed as the number of spots per million PBMCs. Student's t-test was used for comparison of means between experimental groups. Differences that had p values smaller than 0.05 were considered statistically significant.

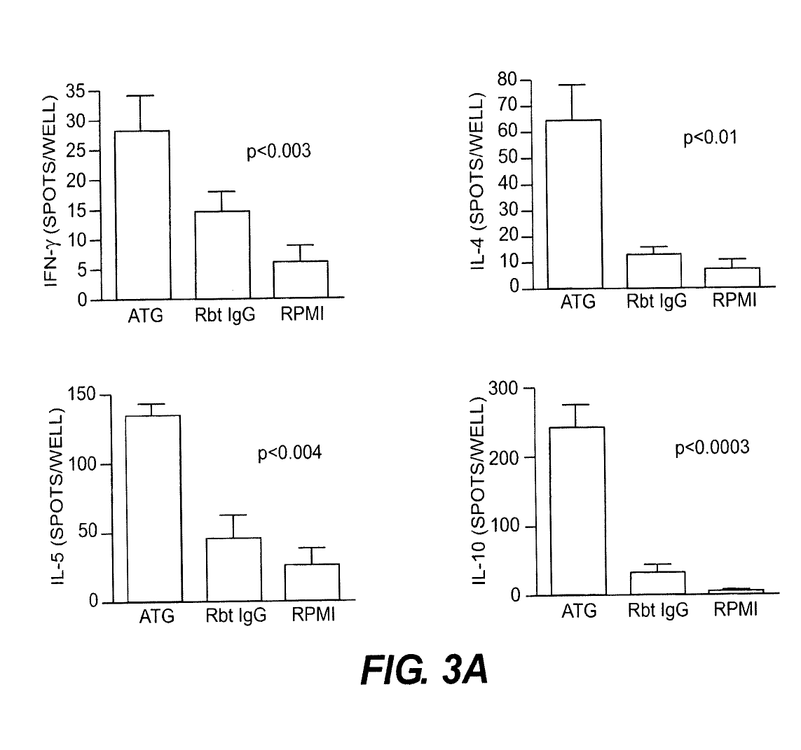

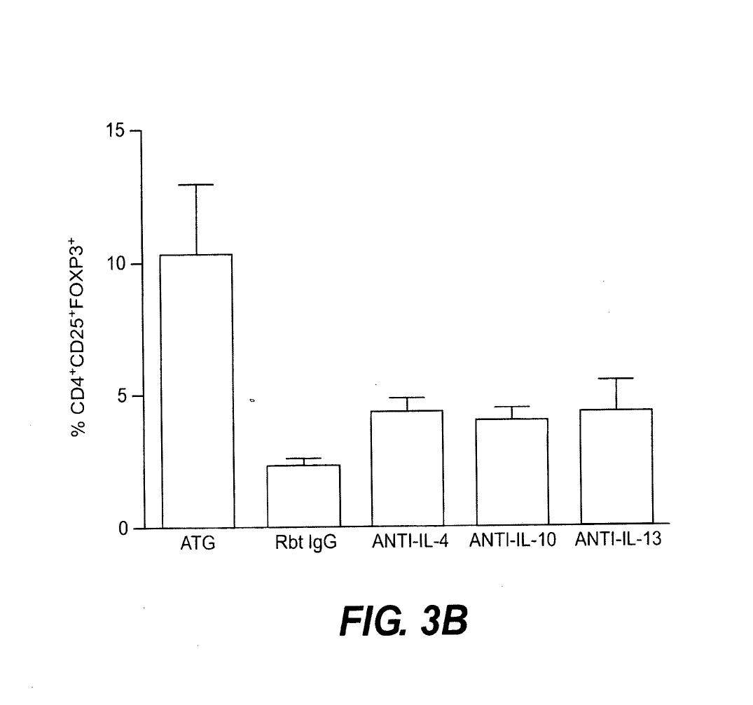

As shown in FIG. 3A, expansion of CD4⁺CD25⁺ T cells by ATG was accompanied by a significant increase in production of IL-4 (64.5±-34.1% vs. 13±9.5%, p=0.01), IL-5 (137±19.7% vs. 45.8±46.7%, p=0.004) and IL-10 (247.8±65.9% vs. 30.2±20.5%, p=0.0003, n=3). Even though the frequency of IFN-γ-producing cells was overall low, it was nevertheless slightly higher in ATG-treated cells relative to control (28.7±20.22 vs. 14.6±10.3, p=0.003, n=4). The production of IL-13 was also higher, however this difference was not statistically significant.

Supernatants from generating cultures (serum-free medium) were tested for the presence of TGF-β using Luminex 100™ system with Beadlyte human multi-cytokine Beadmaster™ kit and Beadlyte human TGF-β1/β2 detection system (Upstate, Charlottesville, Va.) as per manufacturer's protocol.

There was no statistically significant difference in the amounts of secreted TGF-β1 or TGF-β2 between the ATG-treated and control cultures.

To confirm the functional role of Th2 cytokines in expansion of CD4⁺CD25⁺ T cells (Skapenko et al., J. Immunol. 175:6107-6116 (2005)), the generating cultures were incubated with anti-IL-4, anti-IL-13 or anti-IL-10 antibody (10 μg/ml) and analyzed for the expression of FOXP3 by CD4⁺CD25⁺ T cells. The cytokine antibodies were purchased from BD Bioscience (San Jose, Calif.). Results of a representative experiment are shown in FIG. 3B.

### Example 5

### ATG-Expanded Regulatory T Cells Suppress Responder Cells In Vitro

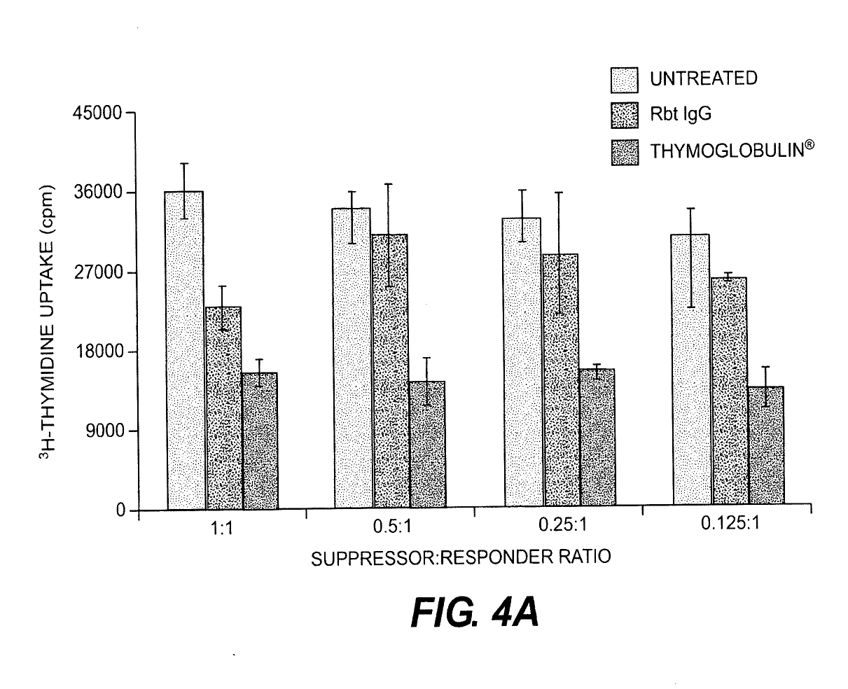

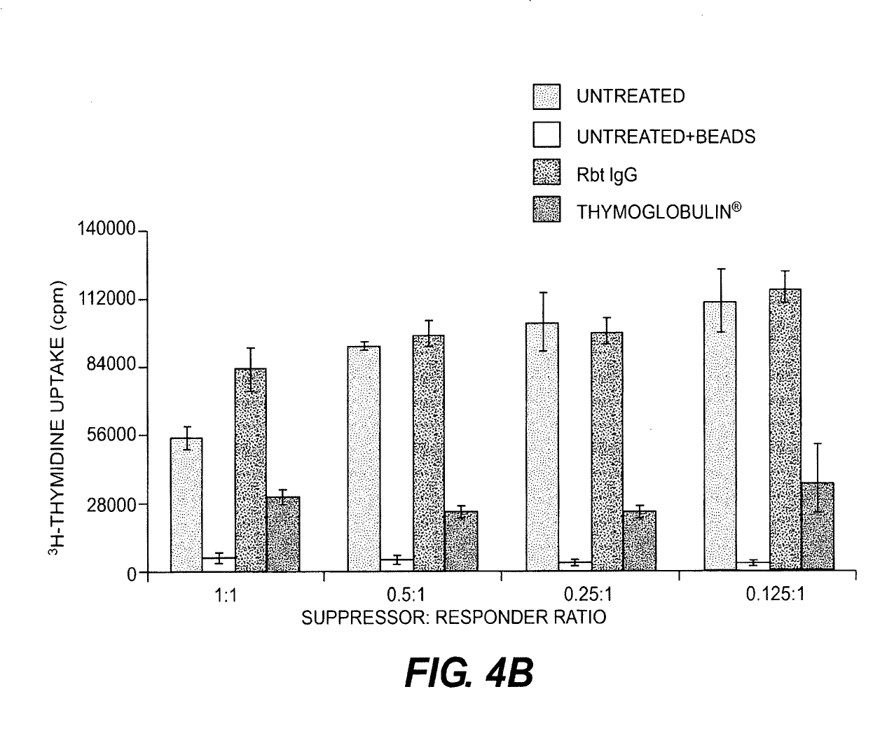

Autologous (to suppressors) PBMCs were thawed, washed and added to wells at 2×10⁵ cells/well. Thymoglobulin®-generated T regulatory cells were washed and added to the appropriate wells giving a final ratio of suppressor to effector cells of 1:1 (2×10⁵ cells/well), 0.5:1 (1×10⁵ cells/well), 0.25:1 (5×10⁴ cells/well), or 0.125:1 (2.5×10⁴ cells/well). Either allogeneic dendritic cells or anti-CD3/anti-CD28 Dynabeads™ were prepared and added to all wells as stimulators. Cultures were incubated for five days at 37° C. ³H-thymidine was added for the last 16-18 hours. Cells were harvested and analyzed for radioactivity by scintillation counting. In a mixed lymphocyte reaction (MLR) where PBMCs were mixed with allogeneic dendritic cells (FIG. 4A), Thymoglobulin®-generated regulatory T cells were able to suppress an MLR response by 34-54%, depending on suppressor to responder ratios. Similarly, suppression was maintained when PBMCs were stimulated with anti-CD3 and anti-CD28 antibodies (FIG. 4B). Proliferative responses were inhibited by Thymoglobulin®-generated T regulatory cells by 57-76% and the suppression was maintained even at the 0.125:1 ratio of suppressor to responder cells.

### Example 6

### ATG-Expanded Regulatory T Cells Suppress Autologous Responder Cells but not Memory Cells

The ability ATG-generated regulatory T cells to suppress immune response to alloantigens was evaluated in a mixed-lymphocyte reaction (MLR) as follows. Cells obtained from the generating cultures described in Example 1 were co-cultured for 120 hours at a 1:1 ratio with fresh responder cells (autologous or third-party PBMCs) or irradiated stimulator cells in a 96-well plate (96 well Cell Culture Cluster, round bottom culture plate, Costar, N.Y.). The cultures were labeled with ³H-thymidine during the last eight hours of culture (Amersham Pharmacia Biotech). Cells were then harvested and radionuclide uptake was measured using a scintillation counting machine. The ability of regulatory T cells to suppress recall-responses to mumps antigens was tested in a like manner.

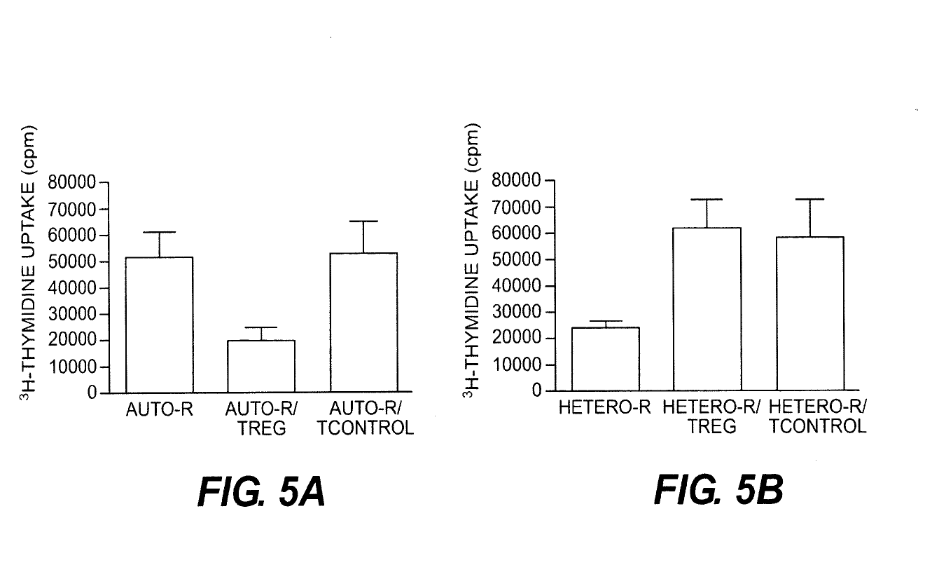

As shown in FIG. 5A, ATG-expanded regulatory T cells (Treg) but not rabbit IgG treated T cells (Tcontrol) significantly suppressed from direct alloimmune responses of autologous responders (Auto-Rs) (61.3±7.4% inhibition by Tregs vs. 20.2±18.4% by rabbit IgG, p=0.01, n=4). ATG-expanded Tregs, however, were unable to suppress the MLR after allostimulation of third-party responder cells (Hetero-Rs) (FIG. 5B).

The proliferative response to recall antigen mumps was not inhibited, indicating that ATG-expanded regulatory T cells did not affect memory cells to the antigen (data not shown).

### Example 7

### ATG Converts CD4+CD25− into CD4+CD25+ T Cells and Promotes Proliferation of CD4+CD25+ Cells

The absolute number of CD4⁺CD25⁺ T cells incubated with ATG, but not rabbit IgG, was dramatically increased after 24 hours of incubation (Table 6). In contrast, the number of CD4⁺CD25⁻ T cells significantly decreased after treatment with ATG as compared with rabbit IgG.

TABLE 6

| | Pre-incubation | | Post-incubation | |
|---|---|---|---|---|
| | ATG | Rabbit IgG | ATG | Rabbit IgG |
| CD4⁺CD25⁺ | 75,833 ± 31,051 | 90,833 ± 33,229 | 639,167 ± 249,448 | 113,333 ± 54,283 |
| CD4⁺CD25⁻ | 1,878,300 ± 322,020 | 1,991,700 ± 379,548 | 1,082,500 ± 301,027 | 1,861,666 ± 238,362 |

The observed expansion of CD4⁺CD25⁺ T cells by ATG may be explained by one or more of following three mechanisms. First, ATG may have preferentially promoted apoptosis of CD4⁺CD25⁻ T cells over CD4⁺CD25⁺ T cells, thereby favoring the latter cells. This possibility is suggested by the published data demonstrating that ATG can in fact induce apoptosis in T lymphocytes via Fas ligand (CD95L) (Genestier et al., Blood 91:2360-2368 (1998); Zand. et al., Transplantation 79:1507-1515 (2005)). Second, ATG may have promoted the proliferation of pre-existing naturally occurring CD4⁺CD25⁺ T cells. Third, ATG may have converted CD4⁺CD25⁻ into CD4⁺CD25⁺ T cells. Each one of these possibilities was further tested.

To evaluate the induction of apoptosis of CD4⁺CD25⁺ and CD4⁺CD25⁻ T cells, PBMCs incubated with ATG or rabbit IgG were stained with antibodies against CD4, CD25, annexin V and 7-amino-actinomycin D (7-AAD) as per manufacturer's instructions (BD Bioscience, San Jose, Calif.). There was no significant difference in apoptosis of CD4⁺CD25⁺ T cells and CD4⁺CD25⁻ T cells incubated for 24 hours with 10 μg/ml of ATG (6.7±3.1% vs. 5±4.7%) or control IgG (5±3% vs. 3.2±2.5%).

Next, the possibility that ATG had a proliferative effect on pre-existing CD4⁺CD25⁺ T cells was addressed. PBMCs were incubated with carboxyfluoroscein succinimidyl ester (CFSE) in the form of 5 mM stock solution in DMSO at final concentration of 1 μM for six minutes at room temperature. CFSE-labeled cells were cultured in vitro with phytohemagglutinin (PHA) (positive control), ATG, and rabbit IgG for 72 hours at 37° C. (Wood et al., Nat. Rev. Immunol. 3:199-210 (2003)). Cells were then stained with anti-human CD4-APC, CD8-PE and CD25-PE. 7-AAD was used to exclude apoptotic cells.

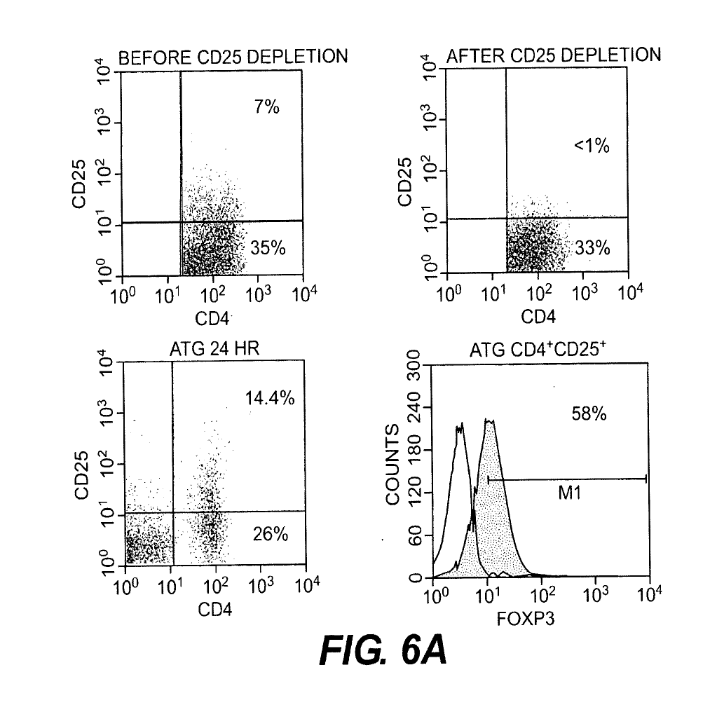

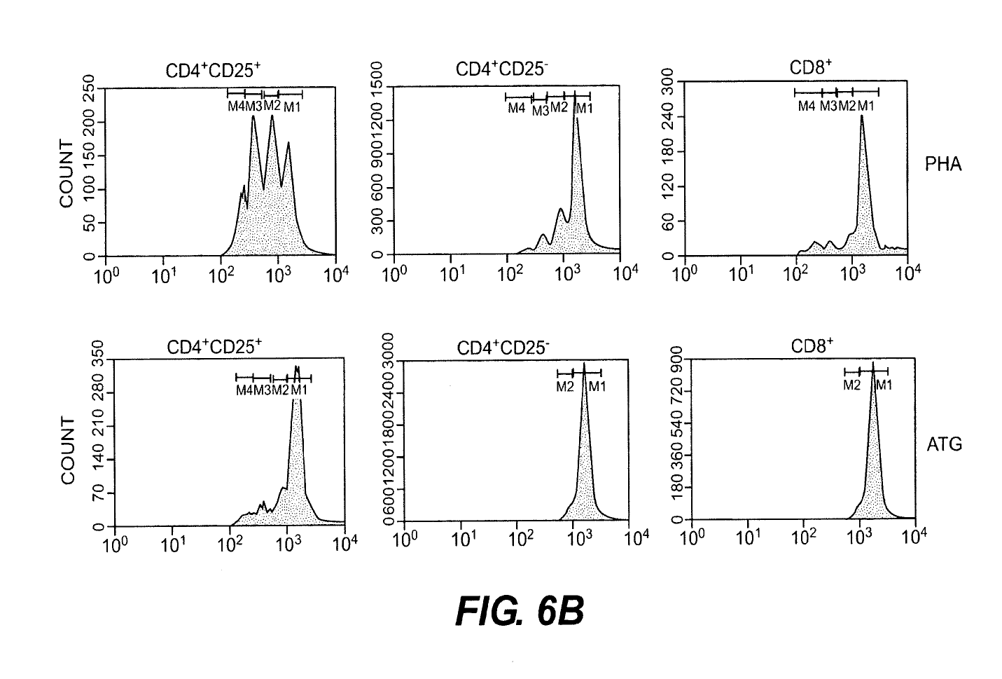

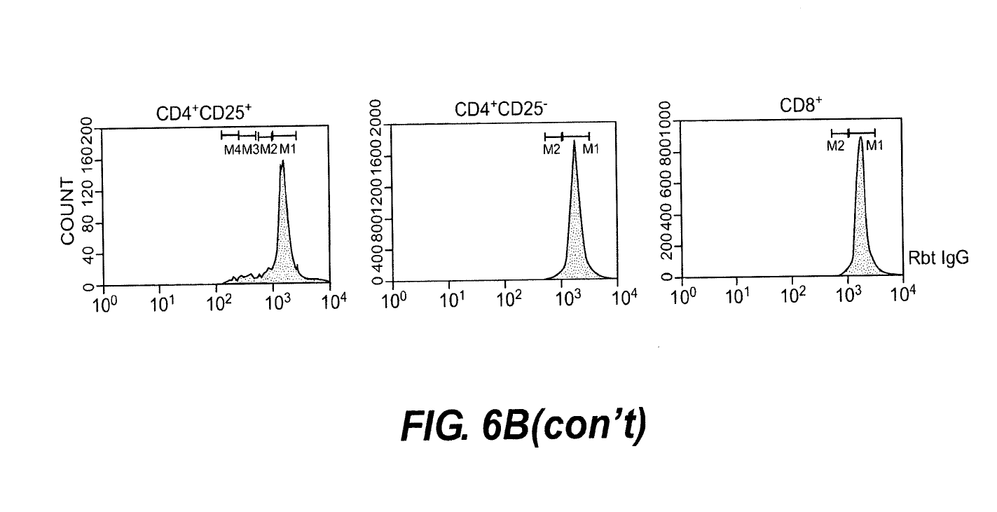

CD8⁺ T cells did not proliferate in the culture incubated with ATG or control IgG (FIG. 6B). Contrastly, 3-4 discrete division cycles of CD4⁺CD25⁻ T cells were observed (proliferative cells, 16±9%, p=0.01, n=3) following treatment with ATG, one division cycle of CD4⁺CD25⁻ T cells under the same conditions was observed, and none in the control (FIG. 6B). In view of a more massive expansion of Tregs observed following treatment with ATG, it is unlikely that proliferation of pre-existing CD4⁺CD25⁺ T cells significantly contributed to the expansion of Tregs.

Finally, to test whether ATG caused conversion of CD4⁺CD25⁻ into CD4⁺CD25⁺ T cells, PBMCs were first depleted of CD25-bearing cells by magnetic cell sorting using MACS columns and MACS separators (Milteny Biotec, Auburn Calif.). CD25-depleted CD4⁺ T cells were then incubated with ATG or rabbit IgG for 24 hours. The cells were then harvested and stained for CD25 and regulatory markers are described in Example 1 and their suppressor activity was assessed as described in Example 4. Results of a representative experiment are shown in FIG. 4. Flow cytometric analysis showed significant up-regulation of CD25 expression on CD4⁺ T cells incubated with ATG but not rabbit IgG (18.7±4% vs. 3.4±1.8%, p=0.02, n=5; see FIG. 6A). The newly generated CD4⁺CD25⁺ T cells expressed FOXP3 at similar levels to that of the pre-existing naturally occurring CD4⁺CD25⁺ T cells expanded with ATG (52.6±13.8% vs. 63.4±12%, p=0.57, n=3; FIG. 6A) and were also capable of suppressing the proliferative response in an MLR (44±14% vs. 5±7%, p=0.01, n=3; FIG. 6B).

### Example 8

### ATG Stimulates Regulatory T Cells in Mice

Mouse splenocytes from C57BLJ6 mice were isolated and cultured at 2×10⁶ cells/ml with 200 U/ml of interleukin-2 and 100 μg/ml of mATG (obtained by immunizing rabbits with mouse thymocytes) or rabbit IgG as a control, at 37° C. with 5% CO₂. Four to five days later, cells were removed from culture and tested for cell surface marker expression and/or immunosuppressive activity.

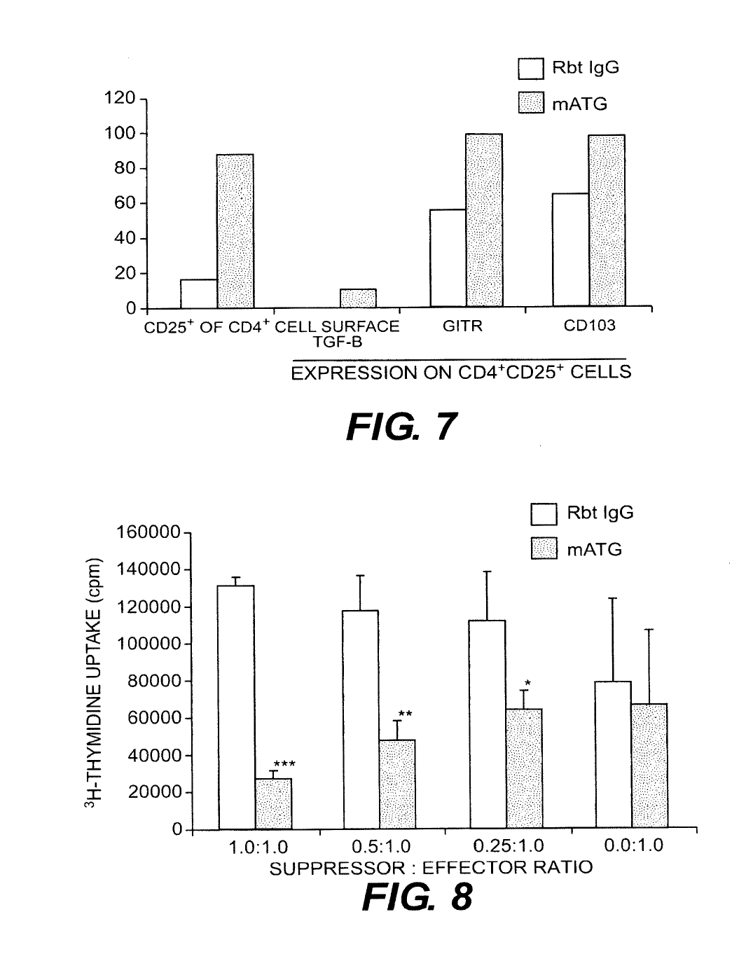

To determine whether stimulation of murine spleen cells resulted in cells with phenotypic properties of regulatory T cells, the cells obtained from the above cultures were surface stained for a variety of markers known to be expressed by Tregs. Cells were first washed with PBS containing 2% fetal calf serum and incubated with fluorescently-labeled antibodies specific for CD4 as well as known markers of regulatory T cells (CD25, GITR and CD103). Surface TGF-β was detected by first incubating cells with an unlabelled chicken anti-TGF-β antibody followed by a fluorochrome-labeled anti-chicken secondary antibody. Combinations of different fluorochrome-conjugated antibodies allowed for detection of these markers specifically on CD4⁺ T cells or CD4⁺CD25⁺ T cells by flow cytometric analysis. Compared to spleen cells stimulated with rabbit IgG, mATG-stimulated cells had higher percentages of CD4⁺ T cells that expressed regulatory T cell markers (see FIG. 7).

To assess whether the mATG-stimulated cells can suppress immune response, normal mouse splenocytes were cultured with T-cell-activating polyclonal antibodies against CD3 and CD28 and in the presence of increasing concentrations of mATG-stimulated spleen cells or control rabbit IgG-stimulated cells, based on a modification of a methods described in Thornton et al., J. Immunol. 172:6519-6523 (2004). Normal splenocytes (effectors) were cultured in 96-well plates at 1×10⁵ cells per well with 5×10⁴ anti-CD3- and anti-CD28-coated beads per well in the presence of increasing ratios of mATG-stimulated spleen cells or control rabbit IgG-stimulated cells (suppressors). Cell cultures were incubated at 37° C. in 5% CO₂ a total of four days with 1 μCi of tritiated thymidine added per well for the last 18 hours of culture. Cells were harvested and tritiated thymidine incorporation measured to detect the level of cell proliferation. A dose-dependent inhibition of proliferative responses was observed in the presence of mATG-stimulated cells, but not with rabbit IgG-stimulated cells (see FIG. 8). These results demonstrate that the cells from mATG-stimulated cultures were able to inhibit ongoing immune responses in vitro.

### Example 9

### ATG Suppresses a Graft-Versus-Host Reaction In Vivo

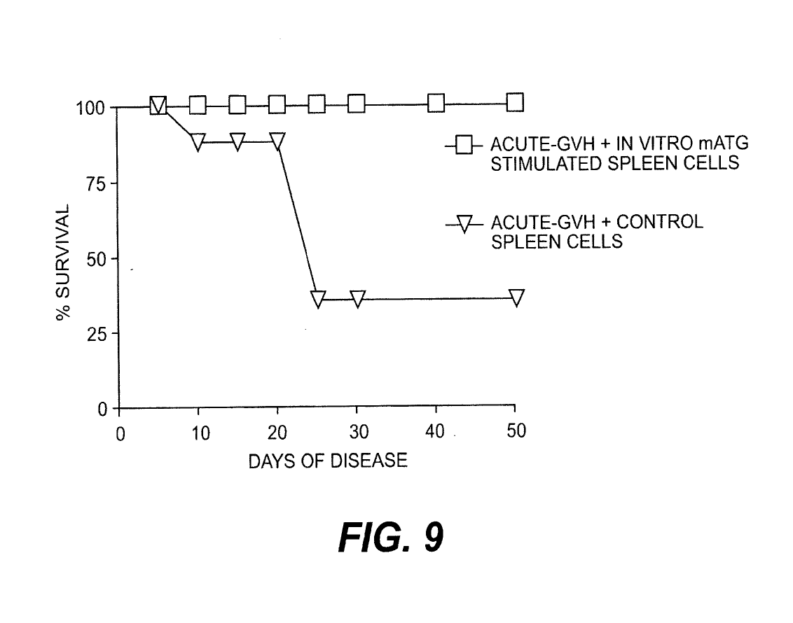

To determine whether adoptive transfer of the in vitro mATG-stimulated spleen cells into mice with graft-versus-host disease (GVHD) would inhibit the disease, cells from mATG-stimulated cultures were collected after five days and injected intravenously into mice induced for a graft-versus-host reaction (allogenic spleen cell transfer). The GVHD model used was a modification of the model described in Li et al., Eur. J. Immunol. 31:617-624 (2001). A splenocyte suspension from donor C57BL16 mice was prepared and injected intravenously into recipient immunodeficient BALB/c mice (RAG-2 knock-out mice that lacked T and B cells). The immunodeficient recipient mice did not require irradiation to eliminate the immune response against the donor cells and a profound acute GVHD was elicited. The transfer of mATG-stimulated spleen cells resulted in protection against the lethality associated with acute graft-versus-host disease (FIG. 9).

These results indicate that murine ATG treatment of normal mouse splenocytes in vitro generates T cells that express markers of regulatory T cells and that these cells are immunosuppressive in vitro and in vivo.

### Example 10

### Role for Autologous Antigen Presenting Cells in ATG-Mediated Treg Expansion

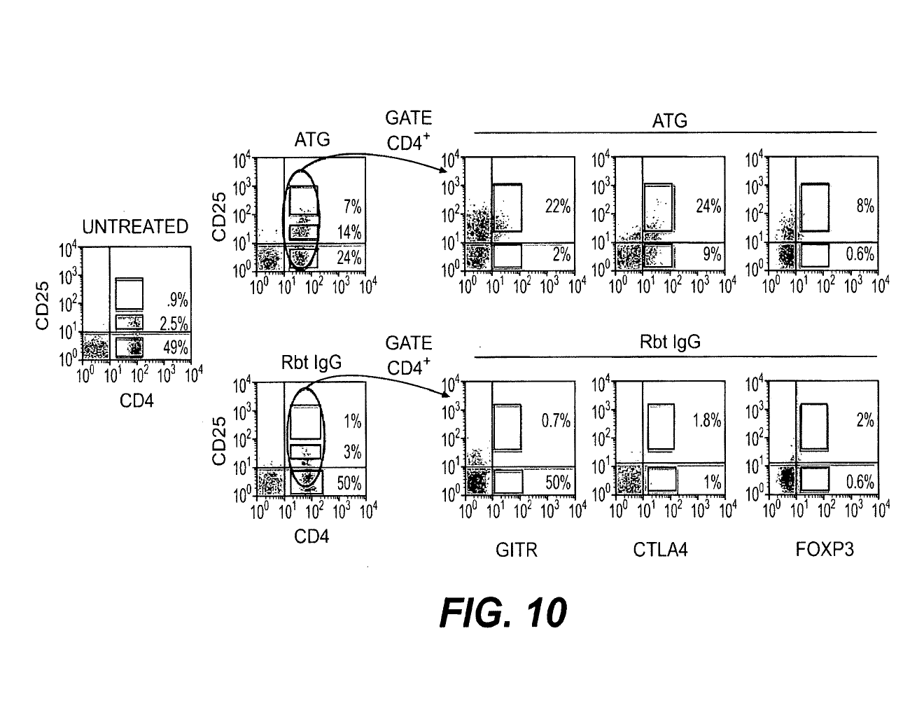

Unless otherwise noted, all methods (e.g., MLR reactions, antibody staining, magnetic cell sorting, and flow cytometry) were performed as previously indicated. As seen in FIG. 10, ex vivo culture of PBMCs from healthy volunteers in the presence of alloantigen (irradiated PBMCs, in a 1:1 ratio) and ATG (10 μg/ml Thymoglobuin®; FIG. 10, top row) but not rabbit Ig (Rbt Ig; FIG. 10, bottom row) for 24 hours triggers significant expansion of CD4⁺CD25⁺FOXP3⁺ Tregs (10.5±5 vs. 3.5±0.9%, p=0.0003; n=5).

These Tregs can efficiently suppress an allogeneic MLR of the original responder cells to donor alloantigens (irradiated PBMCs in a 1:1 ratio; 46±22% inhibition vs. 7±2.8%, p<0.0001; n=9).

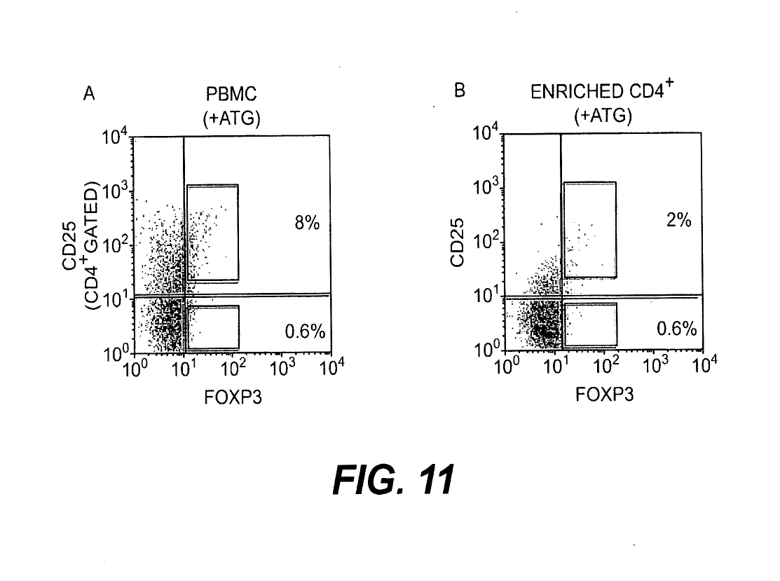

To evaluate the role of APCs in Tregs generation, CD4⁺ T cells were enriched from PBMCs by negative selection on a MACS column using Human CD4⁺ T cell isolation Kit II (Cat. No. 130-091-155, Miltenyi Biotec; percentage of purity 88%±4, n=6). These cells were then incubated with 10 μg/ml ATG (Thymoglobuin®) or Rbt Ig. In contrast to whole fraction PBMCs (FIG. 11A), enriched CD4⁺ T cells did not show expansion of CD4⁺CD25⁺FOXP3⁺ Tregs in the presence of ATG (3±0.7 vs. 9.2±3.8%, p=0.01; n=5; see FIG. 11B).

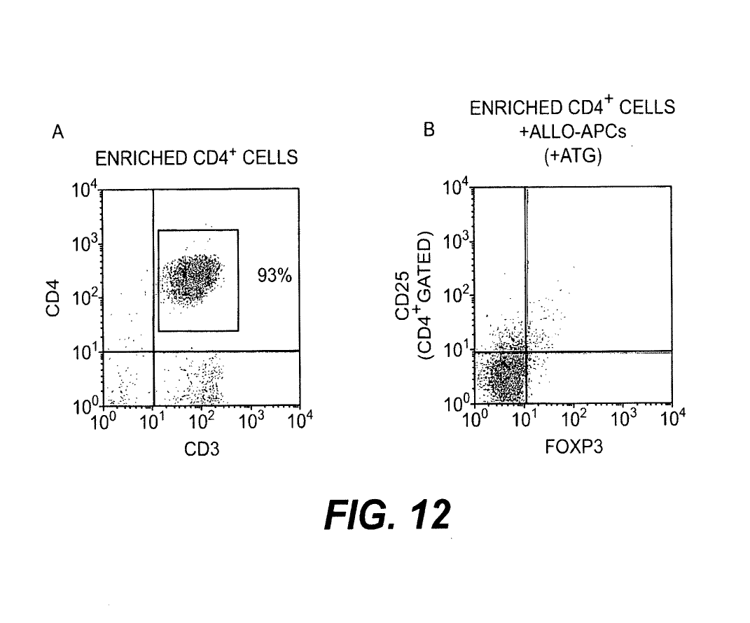

APCs isolated from allogenic PBMCs were added in a 1:1 ratio to CD4⁺ T cells (enriched by negative selection, as above; see FIG. 12A). The addition of allogenic APCs did not expand Tregs (3±1.5 vs. 9.2±3.8%, P=0.02, n=4, see FIG. 12B), demonstrating that unlike autologous APCs, allogenic APCs fail to promote the expansion of Tregs in CD4⁺ cells treated with ATG.

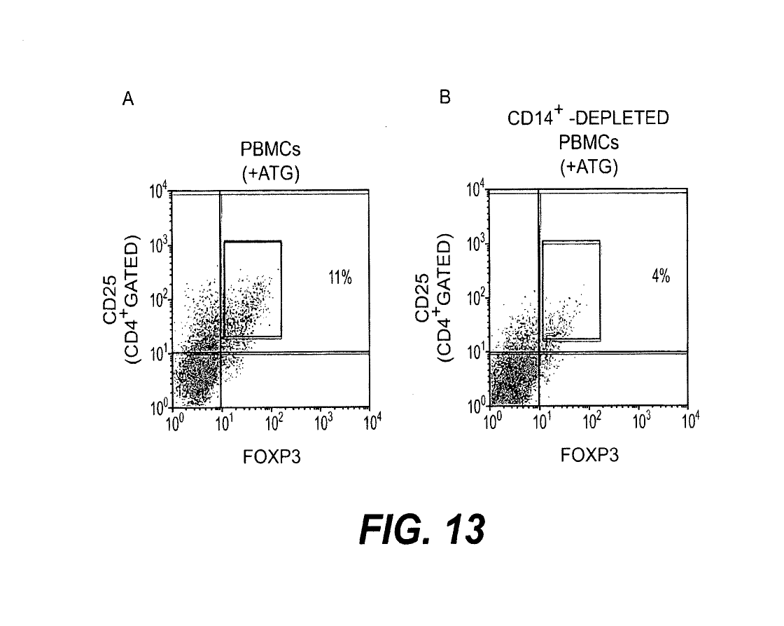

PBMCs were depleted of B cells (CD19⁺) or monocytes (CD14⁺) ex-vivo before incubating with ATG (10 μg/ml Thymoglobuin®) by MACS (Human CD19 Microbeads Cat. No. 130-050-301, and Human CD14 Microbeads Cat. No. 130-050-201, Miltenyi Biotec). While PBMCs depleted of CD19⁺ cells preserved the expansion of Tregs in response to treatment with ATG (6.2±1.8% vs. 9.2±3.8, p=ns, n=4), depletion of CD14⁺ cells abrogates this process (4.2±0.3%, see FIG. 13B). Rbt Ig did not expand Tregs in any of above experiments (3.6±1.2%).

All publications, patents, patent applications, and biological sequences cited in this disclosure are incorporated by reference in their entirety.

1. A method of treating a mammal, comprising:
(a) culturing T cells in the presence of an effective amount of anti-thymocyte globulin (ATG) or an ATG-like composition for a period of time sufficient to generate regulatory T cells; and
(b) administering the regulatory T cells to the mammal.

2. The method of claim 1, further comprising the steps of:
(a) obtaining peripheral blood mononuclear cells (PBMCs) from the mammal;
(b) culturing the PMBCs or a fraction thereof comprising T cells in the presence of an effective amount of ATG or an ATG-like composition for a period of time sufficient to generate regulatory T cells; and
(c) administering the regulatory T cells to the mammal.

3. The method of claim 1, wherein prior to the administration to the mammal, the T cells are cultured in the presence of an effective amount of TGF-β.

4. The method of claim 1, wherein the regulatory T cells are CD4⁺CD25⁺.

5. The method of claim 1, wherein the ATG and ATG-like composition are present at a combined concentration between 0.1 μg/ml and 1 mg/ml.

6. The method of claim 1, wherein the ATG is Thymoglobulin, which is present in the culture at the concentration of 10-50 μg/ml.

7. The method of claim 1, wherein the cultured cells are human.

8. The method of claim 1, wherein the cells are cultured with the ATG or ATG-like composition for at least 8 hours.

9-12. (canceled)

13. The method of claim 1, wherein the mammal is human.

14. The method of claim 1, wherein the treated mammal has or is at risk for an immune-mediated condition.

15. The method of claim 14, wherein the immune-mediated condition is organ or tissue rejection.

16. The method of claim 14, wherein the immune-mediated condition is graft-versus-host disease.

17. The method of claim 14, wherein the immune-mediated condition is an autoimmune disease.

18. The method of claim 14, wherein the ATG is selected from the group consisting of Atgam, ATG-Fresenius S, Tecelec, and Thymoglobulin.

19. A method of making regulatory T cells, comprising culturing T cells in the presence of ATG or an ATG-like composition at a concentration of 1-50 μg/ml for a period of time sufficient to generate the regulatory T cells.

20. The method of claim 19, wherein the ATG is Thymoglobulin.

21-24. (canceled)

25. A method of treating a mammal, comprising:
(a) culturing T cells obtained from a mammal in need of treatment in the presence of an effective amount of anti-thymocyte globulin (ATG) or an ATG-like composition for a period of time sufficient to generate regulatory T cells; and
(b) depleting the circulating lymphocytes of the mammal; and
(c) administering to the mammal the regulatory T cells produced in step (a).

26. The method of claim 25, wherein the circulating lymphocytes are depleted by administering ATG or an ATG-like composition.

27. A method of treating a mammal, comprising:
(a) culturing T cells obtained from a mammal in need of treatment in the presence of an effective amount of anti-thymocyte globulin (ATG) or an ATG-like composition for a period of time sufficient to generate regulatory T cells; and
(b) administering ATG or an ATG-like composition to the mammal, at a dose of less than 1 mg/kg per day; and
(c) administering to the mammal the regulatory T cells produced in step La).

28. The method of claim 27, wherein steps (b) and (c) are performed concomitantly.

29. The method of claim 25 or 27, wherein the T cells are obtained from peripheral blood mononuclear cells (PBMCs).

30. The method of claim 1, 25, or 27, wherein the T cells are obtained from a fraction of peripheral blood mononuclear cells (PBMCs) containing autologous monocytes or dendritic cells.
